(19) United States
(12) Patent Application Publication
(10) Pub. No.: US 2023/0221332 A1
(43) Pub. Date: Jul. 13, 2023

(54) EYE DISEASE EVALUATION METHOD

(71) Applicants: SHIMADZU CORPORATION, Kyoto-shi, Kyoto (JP); KYOTO PREFECTURAL PUBLIC UNIVERSITY CORPORATION, Kyoto-shi, Kyoto (JP)

(72) Inventors: Morio UENO, Kyoto-shi (JP); Junji HAMURO, Kyoto-shi (JP); Kengo YOSHII, Kyoto-shi (JP); Chie SOTOZONO, Kyoto-shi (JP); Kazuhiro SONOMURA, Kyoto-shi (JP); Taka-Aki SATO, Kyoto-shi (JP)

(73) Assignees: SHIMADZU CORPORATION, Kyoto-shi, Kyoto (JP); KYOTO PREFECTURAL PUBLIC UNIVERSITY CORPORATION, Kyoto-shi, Kyoto (JP)

(21) Appl. No.: 18/008,983

(22) PCT Filed: Jun. 7, 2021
(86) PCT No.: PCT/JP2021/021527

§ 371 Date: Dec. 8, 2022

(30) Foreign Application Priority Data
Jun. 9, 2020 (JP) 2020-099804

(51) Int. Cl. G01N 33/68

(52) U.S. Cl. CPC G01N 33/6848; G01N 33/6815

(57) ABSTRACT

A method for diagnosing the risk of glaucoma development, which can diagnose the risk of glaucoma development at the pre-disease stage, and to provide a new method to diagnose disease progression in the end-stage glaucoma. The method can include, for example, a diagnosing method for the risk of an ocular disease development or for the disease progression of an end-stage ocular disease by molecular profile analysis of the ocular anterior tissue microenvironment, comprising a step of measuring the concentration level of a metabolite appearing in the collected aqueous humor specimens by mass spectrometry (MS).

### CROSS-REFERENCE TO RELATED APPLICATIONS

This application claims the benefit of Japanese Patent Application No. 2020-99804, filed with the Japan Patent Office on Jun. 9, 2020. The Japanese application is hereby incorporated by reference for all purposes as if the entire application documents (specification, claims, drawings, and abstract) were expressly set forth herein.

### TECHNICAL FIELD

The present invention belongs to the technical field of medical diagnosis of ocular diseases, especially ocular diseases caused by disruption of the anterior chamber microenvironment, and more specifically, corneal endothelial failures and pathologically related diseases commonly referred to as glaucoma. In the technical field, in addition to diagnose the molecular profiles of proteinaceous molecules, nucleic acid, and extracellular vesicles etc. that appear in the ocular anterior tissue microenvironment, especially in the aqueous humors, the diagnosis of the concentration levels of metabolites in the aqueous humors by mass spectrometry (MS) will be provided to accomplish the present invention; the present invention relates to a method for diagnosing the risk of developing ocular diseases associated with the visual impairment of the ocular anterior tissue microenvironment or diagnosing the risk of developing glaucoma or diagnosing the pathological progression of glaucoma, characterized by a comprehensive molecular dynamics analysis technique that can only be possible by measuring the concentration levels of metabolites by mass spectrometry (MS), together with proteinaceous molecules, nucleic acid molecules, and extracellular vesicles etc. that appear in the ocular anterior tissue microenvironment.

### BACKGROUND OF THE INVENTION

The provision of appropriate medical care is a fundamental social issue for sensory organ disorders, as well as for cancer, cardiovascular disorders, and cognitive dysfunction, in countries worldwide including Japan, which is a super-aging society. Eighty percent of sensory information comes via the visual system, which is the foundation to establish the individual dignity. There is no doubt that environmental factors, in addition to genetic factors, are involved in age-related ocular diseases, especially in glaucoma. In fact, environmental factors are also known to influence retinal ganglion cells/trabecular meshwork function/intraocular pressure (TOP), etc., as the pathophysiology of glaucoma. The present inventors have conducted research to find genes responsible in the development of various type glaucoma, both independently and through participating in international consortia. However, with the exception of LOXL1, the relative risk of the identified genes was turned out low. Consequently, the pathogenesis has not been elucidated, and the functions of the genes identified as risk factors and the association of them with clinical glaucoma types have not been determined. In other words, the usefulness of these genes has not been proven at all.

Therefore, the present inventors have turned their scientific focus to environmental factors in general, including aging, and have hypothesized the existence of “environmental factors” that impair the ocular anterior tissue homeostasis in addition to genetic elements. First of all, the present inventors have been challenging the analysis of the molecular profiles and the identification of the relevant molecules in the anterior chamber microenvironment, and the analysis of functional network composition among ocular anterior tissues for many years.

There are many ocular diseases that can be caused by impairment of the homeostasis of ocular anterior tissue microenvironment. The most common of them are corneal endothelial failures, glaucoma, and cataracts. All of these diseases can occur secondary to trauma, ophthalmic surgery, uveitis, or other diseases that cause a disruption of the blood-aqueous barrier.

Along line with this notion, the present inventors have been intensively conducting researches, for many years, on the molecular dynamics of ocular anterior tissue microenvironmental factors in corneal endothelial failures, glaucoma, and cataract, and on the molecular profiles of proteinaceous molecules, nucleic acids, extracellular vesicles, exosomes, and metabolites that appear in the aqueous humors, in order to associate the pathological characteristics of these factors with individual ocular disease state. Then, we have succeeded to establish a diagnosing technique for selecting disease-type- and pathological-progression-state-specific molecular profiles, and have completed the invention herein described.

The reason for focusing on ocular anterior tissue microenvironmental factors is because the analysis of only systemic factors, such as risk genes, will not specify the function of locally functioning exacerbating factors. Several body fluids are known to be applicable to the diagnosis of pathogenic risk or disease progression, including blood, urine, saliva, and sweat, and in the field of ophthalmology, tear fluid is also a target. However, in diseases in which localized pathologies are exacerbated or regulated, local molecular dynamics are not necessarily reflected to the dynamics of systemic body fluids. Even in the field related to the present invention, there are only a few prior findings related to blood and tear fluid, but none of them have been confirmed to be useful at the level of practical clinical application.

The present inventors have long conducted clinical research on regenerative medicine using cultured human corneal endothelial cells with cell phenotypes homologous to those of in vivo tissues, and have confirmed clinical POC (Proof of Concept). Cultured human corneal endothelial cells with various cell phenotypes can be obtained according to various culture environments. That can be regarded as a simulation model of the effects of culture environment factors on cell phenotypes and cell degeneration in vitro. The microenvironment of corneal endothelial tissue, particularly the ocular anterior tissue microenvironment, is as diverse as the culture environment, and the in vitro experimental results indicated that ocular anterior tissue a microenvironment is presumed to induce aging, degeneration, and transformation of cells constituting human corneal endothelial tissue. Bullous keratopathy, a corneal opacity caused by corneal endothelial tissue failures, is associated with severe visual impairment and long-term disability on life. Although intractable, the cause and pathophysiology of bullous keratopathy are still unknown, and there is so far no drug therapy available. In Japan, approximately 800 cornea transplantation are performed annually on patients with bullous keratopathy, but the failed tissue is only replaced by donor tissue, resulting in a persistent decrease in endothelial cell density (late corneal tissue failures) after the transplantation procedure. It remains unclear whether the pathophysiology of tissue dysfunction is homogeneous or diverse, and what causes late corneal tissue failures.

Under these background, in order to provide optimal corneal transplantation and regenerative medicine for bullous keratopathy patients, the present inventors have been conducting researches with the aim of establishing an innovative molecular diagnostic method and utilizing it as an indicator for standardization of medical care by analyzing the ocular anterior tissue microenvironment of bullous keratopathy patients, particularly microRNAs (miRNAs), exosomes, extracellular vesicles (EVs), and SASP (senescence-associated secretory phenotype)-related cytokines in the aqueous humors and, in addition, various metabolites that can be measured by mass spectrometry. This research is aimed at establishing a method for diagnosing the risk of developing ocular diseases related to the impairment of the ocular anterior tissue microenvironment and the pathological progression of ocular diseases, especially for diagnosing the risk of glaucoma development and the pathological progression of glaucoma, which can be achieved only by comprehensive molecular dynamics analysis technology.

The reason for focusing on the analysis of these molecular dynamics may be that these molecules form a network among the ocular anterior tissues, as shown in FIG. 2. The analysis of the network among the ocular anterior tissues has made it possible for the first time to provide a technique for diagnosing the risk of ocular disease development or for diagnosing the risk of ocular disease development due to the impairment of homeostasis of ocular anterior tissue microenvironment, and more especially diagnosing the pathogenesis of the ocular disease referred to as glaucoma, by targeting the molecular dynamics of the ocular anterior tissue environment, which is directly related to the pathogenesis of these diseases.

As one of the ocular anterior tissue microenvironmental factors addressed by the present invention, the present inventors will give an introduction of a part of the network formed among ocular anterior tissues, pertaining to molecular profiles in aqueous humors.

The blood-aqueous barrier, which is controlled by ciliary achromatic epithelial cells and iris vascular endothelial cells, limits the traffic of molecules with high molecular weight from plasma to aqueous humor. In fact, the protein concentration in the aqueous humors is as low as 25 mg/dL, whereas that in the plasma is 6 g/dL. However, when the blood-aqueous barrier is injured by trauma or inflammation, high molecular weight proteins such as immunoglobulins and β-lipoproteins also leak from blood and appear in the aqueous humors. Thus, external stresses such as trauma and inflammation are among the local factors that can impair the homeostasis of ocular anterior tissue microenvironment. The noise caused by these disturbing factors must be fully considered when establishing the diagnosing technique as the purpose of the present invention.

This clearly indicates that the molecular profiles of the aqueous humors are reversibly or irreversibly variable and impaired in ocular inflammatory diseases, including cataracts, and that care must be taken in selecting the control aqueous humors to be used for analysis of the molecular profiles of the aqueous humors and for identification of candidate molecules to be used as diagnosis indices, depending on the target disease handled with.

In the present invention, in the molecular profile analysis of aqueous humors and the identification of candidate molecules to be used as diagnosis indices, cataract patients were selected as controls instead of normal subjects, with the intention of excluding nonspecific leakage of molecules associating with inflammation. By doing this, the present inventors have first completed a disease-specific and practical invention, unlike previous reports. We have confirmed that the profiles of not only cytokines as proteinaceous molecules but also miRNAs and metabolites in the identification of candidate molecules to be used as diagnosis indices aqueous humors are different in cataract patients than in patients with corneal endothelial failures (FIGS. 3 to 5).

There is a possibility that variations exist in molecular dynamics of the ocular anterior tissue microenvironment according to the following cascade pathway; corneal endothelial cell degeneration (due to factors such as aging and oxidative stress)→changes in the molecular species and amounts produced by these cells→degeneration and cell death of neighboring trabecular meshwork cells→decreased function of trabecular meshwork→increased intraocular pressure→glaucoma (see FIG. 6). Based on the following points, it is clinically presumed that most cases of glaucoma that develop after corneal transplantation are steroidal glaucoma. (1) When all-layer corneal transplantation was the only option for corneal failures in the past, the use of steroids after surgery was prolonged, and the prevalence of elevated intraocular pressure was high. (2) With corneal endothelial transplantation (DSAEK), the postoperative steroid use was reduced and shortened, and the prevalence of elevated TOP was drastically reduced. (3) The combined use of oral administration of an immunosuppressant drug (cyclosporine) led to the dose reduction of steroid, and the prevalence of TOP elevation decreased. (4) Trabecular incision, which is a successful treatment for steroidal glaucoma, is often successful in treating elevated intraocular pressure after corneal transplantation.

However, in glaucoma/corneal endothelial failures secondary to exfoliation syndrome, changes in aqueous humor composition due to the injury of the blood-aqueous barrier are considered common to both diseases. The mechanism of the development of glaucoma; TOP elevation→corneal endothelial failures→glaucoma (TOP elevation) is currently unexplainable from clinical observations in that corneal endothelial failures and glaucoma do not necessarily occur simultaneously in patients with exfoliation syndrome, and basic molecular dynamics analysis is required (The present invention provides and discloses a means to solve this problem in part.).

The mechanism by which exfoliation syndrome is complicated with glaucoma is thought to involve dysfunction of trabecular meshwork cells which is caused by phagocytosis by trabecular meshwork cells of pigment granules, which are released from iris pigment epithelial cells through the deposition of exfoliative material in the trabecular meshwork tissue, production of exfoliative material in the trabecular tissue, and then the degeneration of iris pigment epithelial cells, and which then arrive in trabecular meshwork tissue with the aqueous humor flow. In addition, abnormal thickening of Descemet's membrane and production of exfoliative material by corneal endothelial cells are thought to be involved in corneal endothelial failures (cell loss and cell polymorphism) associated with exfoliation syndrome, but at present, no knowledge has been obtained at a level that can be immediately applied to diagnosing techniques.

One more point to keep in mind in relation to the present invention is the background factors of the patients from whom the specimens are collected. Along with the background factors such as age, gender, and complications, the quality of medical interventions used in diseases related to ocular anterior tissue dysfunction is also important.

Common/individual mostly performed medications to three forms of glaucoma include sympathetic blocking drugs, prostaglandin derivatives, carbonic anhydrase inhibitors, parasympathomimetic drugs, sympathomimetic drugs, and Rho kinase inhibitors as an eyedrop medicine, and carbonic anhydrase inhibitors (taken with potassium L-aspartate to prevent hypokalemia) as an oral medicine for the main purpose of TOP lowering; methylcobalamin (a type of vitamin B12) as an oral medicine for the purpose of nerve activation; and steroids used in corneal transplantation for corneal endothelial failures. It is one of the essential elements of the present invention to take into account the differences in these background medication factors to the extent possible, and to depict disease-specific molecular dynamic anomalies in the ocular anterior tissue environments.

Glaucoma is an ocular disease in which the retinal nerve fiber layer and optic nerve are damaged from retinal ganglion cell death or their axonal damage, resulting in visual field loss. Glaucoma is a neurodegenerative disease, the progression of which is irreversible and often occurs gradually without subjective symptoms. The progression of glaucoma leads to blindness through the “very early stage with no detectable changes” (pre-disease stage), the “early stage with no subjective symptoms”, and the “visual impairment stage”. Early detection, and early treatment and management are therefore important.

Glaucoma is the leading cause of visual impairment in Japan (25%, 50,000 people), and its prevalence is estimated to be about 5% in people over 40 years of age. Glaucoma is also one of the leading causes of blindness worldwide.

The etiology of glaucoma includes an increase in the pressure inside the eyebowls (intraocular pressure), but there is glaucoma without the increase in intraocular pressure. Therefore, it is difficult to correctly diagnose whether or not a person has glaucoma, solely by measuring intraocular pressure, but it is clearly a risk factor. And because of the possibility of quantitative measurement, intraocular pressure measurement is widely used in the diagnosis of glaucoma.

Glaucoma includes primary open-angle glaucoma (POAG), primary angle closure glaucoma, secondary glaucoma, and developmental glaucoma. Primary open-angle glaucoma with intraocular pressure in the normal range (20 mmHg or less) is called normal tension glaucoma (NTG). Whereas primary open-angle glaucoma with an intraocular pressure greater than 20 mmHg is called primary open-angle glaucoma in the narrow sense. More than 70% of Japanese glaucoma patients have NTG, but since their intraocular pressure is normal, it is not possible to distinguish this type of glaucoma by intraocular pressure testing alone.

There is pseudoexfoliative glaucoma (PEG), which is caused by deposits of pseudoexfoliative material in the trabecular meshwork, iris, or anterior capsule of the lens (pseudo desquamous syndrome), or there is also refractory glaucoma called pseudoexfoliative glaucoma/pseudoexfoliative syndrome (PEX).

The disease types are assumed to have a large deviation from the normal profile of cytokines, miRNAs, exosomes, and metabolites in the aqueous humors in the order of exfoliative glaucoma/exfoliative syndrome>narrow primary open-angle glaucoma>normal tension glaucoma. Two pathways are assumed to be involved in the molecular profile in the aqueous humors and glaucoma pathogenesis. That is, the two pathways are broadly divided into those related to elevated intraocular pressure and those related to vision disorder, and we believe that there is a difference in the deviation of molecular dynamics of the ocular anterior tissue microenvironment.

Since the vulnerability of the optic nerve plays a major role in the pathogenesis of normal tension glaucoma, normal tension glaucoma specimens were applied in this invention, considering suitable for the identification of candidate molecules for the diagnosis of future development of glaucoma at the very early stage without any clinical sign of disease.

In addition to intraocular pressure testing, a method of diagnosing the risk of glaucoma development or diagnosing the disease progression involves the optical coherence tomography method (OCT), which takes fundus photographs. However, OCT cannot be used for diagnosing at the “very early stage with no detectable changes of fundus” or for diagnosing the disease progression when it has progressed to an extremely advanced stage.

In addition to intraocular pressure, other factors participating the pathogenesis of glaucoma are said to include poor optic nerve blood flow, neurotoxicity, neurotrophic factor depletion, and oxidative stress. Therefore, it is presumed that these factors are reflected systemically, and the possibility of analyzing metabolomics in blood/plasma as a tool to diagnose the risk of glaucoma development or the progression of the disease has been investigated (Non-Patent Documents 1 and 2). According to Non-Patent Document 1, metabolites in plasma were analyzed using LC/MS and it was found that POAG patients showed the significant difference in the amounts of palmitoylcarnitine, sphingolipids, vitamin D-related compounds, and steroid precursors compared with normal subjects. In Non-Patent Document 2, LC/MS was used to identify eighteen discriminant metabolites belonging to carbohydrates, acylcarnitines, phosphatidylcholine, amino acids, and polyamine family members.

Although it is controversial whether glaucoma is a localized or a systemic disease, the present invention targets a pathogenesis that is limitedly localized and is not reflected systemically, i.e., a pathogenesis that is localized an d manifests itself in the ocular anterior tissue microenvironment. The rationales for glaucoma being a systemic disease are: (1) diabetes and hypertension are risk factors for glaucoma; (2) hypotension is also a risk factor for glaucoma (in relation to a decrease in ocular perfusion pressure); (3) age is a risk factor for glaucoma; and (4) BDNF in tear fluid is known to be decreased in patients with normal tension glaucoma.

Conversely, rationales that glaucoma is a localized disease are: (1) ocular connective tissue factors such as high intraocular pressure, myopia, thin cornea, and disc hemorrhage are risk factors for glaucoma; (2) optic nerve cup ping, a characteristic finding of glaucoma, is caused by compression, stretching, or backward bowing of the lamina cribrosa in the optic nerve head area in response to intraocular pressure. For the change of the lamina cribrosa, in addition to simple compression induced by intraocular pressure, damage and remodeling of the lamina cribrosa is closely related. As a result, the pores of lamina cribrosa, through which the axons of retinal ganglion cells pass, is deformed, resulting in axonal transport disruption. This axonal transport disruption is believed to lead to retinal ganglion cell death.

The present invention is the first and original one that could be completed, based on these concepts mentioned above, by focusing on the technology for detecting disruption of the ocular anterior tissue microenvironment, characterized by the “discovery and application of factors disturbing the ocular anterior tissue microenvironment homeostasis, that show specific molecular dynamics locally in the ocular anterior tissue microenvironment”, especially characterized by the comprehensive molecular profile analysis technology that can only be possible by measuring the concentration levels of metabolites detectable only locally in the aqueous humors, in addition to, by testing individually among distinct disease state, the molecular dynamics of proteinaceous molecules, nucleic acid, and extracellular vesicles, that appear in the aqueous humor, unlike those of which changes appear systemically in body fluids such as blood, urine, and saliva.

Non-Patent Document 3 also analyzed inflammatory cytokines in the aqueous humors and showed their association with open-angle glaucoma and other conditions. Specifically, in POAG or PEG, interleukin (IL)-1α, IL-2, IL-4, IL-8, IL-23, and CCL2 levels were significantly increased, among others, suggesting that IL-8 elevation is a risk factor important for both detection and management of glaucoma.

### PRIOR ART

### Patent Document

[Non-Patent Document 1] Burgess L G, et al., Invest Ophthalmol Vis Sci., 2015; 56: 5020-5028.
[Non-Patent Document 2] Leruez S, et al. Invest Ophthalmol Vis Sci., 2018; 59:
[Non-Patent Document 3] Ikuyo Chono, et al, Scientific Reports, (2018) 8:14533.

### SUMMARY OF THE INVENTION

### Problem to be Solved by the Invention

The present invention aims to provide a practical diagnosing technology for the risk of developing and for the advanced pathogenesis of ocular diseases, related to disruption of the ocular anterior tissue microenvironment, that can only be possible by measuring the concentration levels of metabolites, in addition to the measurement of the molecular profiles of proteinaceous molecules, nucleic acids molecules, and extracellular vesicles, by testing each individual disease state, that appear in the ocular anterior tissue microenvironment, especially in aqueous humors targeting at ocular diseases caused by disruption of the homeostasis in ocular anterior tissue microenvironment, especially corneal endothelial failures including bullous keratopathy and Fuchs' corneal dystrophy, and their pathologically related diseases commonly referred to as glaucoma.

Bullous keratopathy, a corneal opacity caused by corneal endothelial failures, is an intractable disease with severe visual impairment, but its cause and pathogenesis are still unclear, and scientific diagnosis methods for determining its medical intervention are poor. The inventors have established the world's first regenerative medicine for tissue reconstitution by injecting cultured human corneal endothelial cells homogeneous in their cell quality as an innovative medical modality applicable to this condition, and have confirmed a good long-term prognosis (Shigeru Kinoshita, et al, New Engl. J Med. 2018; 378 New Engl. J Med. 2018; 378:995-1003.). However, stratification of patients eligible for corneal transplantation and this regenerative medicine has not yet been achieved. In order to provide optimal corneal transplantation and regenerative medicine for patients with bullous keratopathy as a standard of care both domestically and internationally, it is important to establish and standardize a scientific molecular diagnosis method for environmental factors in target patient's graft bed and to establish an all-inclusive registry format. The diversity of ocular anterior tissue microenvironmental factors in patients with bullous keratopathy was exemplarily verified using cytokine profiles as a model (FIG. 1).

The present invention solves similar problems in glaucoma disease as other ocular diseases involving the ocular anterior tissue microenvironment. In particular, the present invention aims to provide diagnosis technology that can predict future disease development in the pre-disease stage, and to create technology to identify pathological progression in patients with the highly developed end-stage disease and high risk of blindness that cannot be determined with current optical coherence tomography technology. The objective also includes providing a technology that enables molecular diagnosis of the differences among the three pathologically distinct types of glaucoma, as well as a new diagnosis technology for the risk of disease development or for the pathological disease progression by clarifying the bidirectional causal relationship between corneal endothelial tissue failures and visual field impairment or intraocular pressure elevation. This will be indispensable for providing the basic technology for innovative drug discovery.

The present invention is based on the originally innovative concept that the dysfunction of predisposition to ocular anterior tissues is ascribed to the aggravating vicious cycle of cellular senescence triggered by the biased molecular composition in the ocular anterior tissue microenvironment such as cytokines, miRNAs, exosomes, metabolites, and other molecules and the molecular groups thereof, and innate inflammation including the complement system.

Basic data on the molecular dynamics of the anterior chamber microenvironment that support the probability of the novel notion of this invention had been being accumulated (proteome analysis, and transcriptome analysis), for example: the expression of miRNAs (miR-34-a and miR-378), which are common to both the dysfunctional tissues of human corneal endothelium and subpopulations of cultured human corneal endothelial cells with cell state transition, is downregulated, and these miRNAs regulate the mitochondrial functional homeostasis and the production of SASP-related cytokines such as IL6, IL8 and VEGF through the epigenetic mechanisms. The present invention will lead to the clarification of molecular pathogenesis and the identification of target molecular species responsible for the pathogenesis, which are essential for drug discovery, from a new standpoint that targets metabolic reprogramming and mitochondrial function instead of the so far applied conventional method of targeting gene expression.

Although early detection, early therapeutic treatment and management are said to be clinically critical in glaucoma, diagnosis at “very early stage with no detectable clinically abnormal changes,” for example, is so far absolutely impossible. In addition, as mentioned above, existing optical methods (fundus photography and OCT) cannot be used for diagnosis at the “very early stage with no clinically abnormal signs”. Currently, the only quantifiable risk factor that can be used in clinics is intraocular pressure, and intraocular pressure testing is certainly important in diagnosing the development and progression of glaucoma. However, intraocular pressure testing is ineffective for normal tension glaucoma (NTG), common form of glaucoma.

In the “visual impairment stage”, visual field testing is used to determine the progression of visual field defects, but it requires many tests before to gain the final confirmative diagnosis, and is stressful for the elderly, for example. In addition, because patients with end-stage glaucoma have poor visual fixation, conventional visual field testing is not reproducible, making it difficult to diagnose disease progression.

Therefore, new clinical diagnosing methods other than or in addition to optical techniques and TOP testing are needed.

The main subject of the present invention is to provide a new method for diagnosing the risk of glaucoma development, which can diagnose the risk of glaucoma development even at the pre-disease stage, namely, at the “very early stage with no detectable clinical abnormal changes”, and to provide a new method to diagnose disease progression in the end-stage glaucoma, namely, the “visual impairment stage”.

### Means for Solving the Problem

The inventors have earnestly analyzed the molecular profiles of the ocular anterior tissue chamber microenvironment and found that the above subjects can be solved by analyzing concentrations of metabolites measurable by mass spectrometry as well as microRNAs, exosomes, extracellular vesicle (EVs) and SASP-related cytokines that appear in the aqueous humors, and completed the present invention.

The present invention can include, for example, the following embodiments.

[1] A diagnosing method for the risk of an ocular disease development or for the disease progression of an end-stage ocular disease by molecular profile analysis of the ocular anterior tissue microenvironment, comprising a step of measuring the concentration level of a metabolite appearing in the collected aqueous humor specimens by mass spectrometry (MS).
[2] The diagnosing method according to the [1] above, wherein the ocular disease is glaucoma.
[3] The diagnosing method according to the [1] or [2] above, wherein the collected aqueous humor specimens are from a subject in a pre-disease stage with no clinically abnormal signs of ocular disease without identification of the pathological characteristics of the ocular disease.
[4] The diagnosing method according to any one of the [1] to [3] above, wherein the mass spectrometry (MS) is gas chromatography-mass spectrometry (GC-MS) and/or liquid chromatography-mass spectrometry (LC-MS).
[5] The diagnosing method according to any one of the [1] to [4] above, wherein the metabolite is contained in extracellular vesicle (EVs) in the aqueous humors.
[6] The diagnosing method according to any one of the [1] to [5] above, further comprising a step of selecting a metabolite that shows different molecular dynamics from that in tear fluid or blood.
[7] The diagnosing method according to any one of the [2] to [6] above, wherein glaucoma is primary open-angle glaucoma (POAG), normal tension glaucoma (NTG), or pseudoexfoliative glaucoma/pseudoexfoliative syndrome (PEG).
[8] The diagnosing method according to any one of the [2] to [7] above, further comprising a step of discriminating the distinct disease pathogenesis peculiar to each of the three types of glaucoma: primary open-angle glaucoma (POAG), normal tension glaucoma (NTG), and pseudoexfoliative glaucoma/pseudoexfoliative syndrome (PEG).
[9] The diagnosing method according to any one of the [2] to [8] above, wherein the metabolite is selected from those common or not common with the bias observed in subjects with corneal endothelial failures.
[10] The diagnosing method according to any one of the [2] to [9] above, wherein the metabolite is at least one selected from the group consisting of sugar or polyol, a product in the citric acid cycle, acylcarnitine, polyamine, amino acid, and cAMP.
[11] The diagnosing method according to the [10] above, wherein the sugar or polyol is arabinonic acid, myo-inositol, or fructose; the product in the citric acid cycle is citric acid or isocitric acid; the acylcarnitine is carnitine, isobutyrylcarnitine (C4), or propionylcarnitine; the polyamine is spermidine; and the amino acid is asy-dimethylarginine, quinolinic acid, cysteine, or 3-methylhistidine.
[12] The diagnosing method according to the [10] or [11] above, wherein the sugar or polyol is arabinonic acid or myo-inositol; the acylcarnitine is isobutyrylcarnitine (C4); and the amino acid is cysteine.
[13] The diagnosing method according to any one of the [2] to [9] above, wherein the metabolite is at least one selected from the group consisting of 2-aminoadipic acid, mannose, GSH (glutathione), alanine, spermine, asparagine, choline, glutamine, glutamic acid, pyroglutamic acid, acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formyl kynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.
[14] The diagnosing method according to any one of the [2] to [9] above, wherein the glaucoma is normal tension glaucoma (NTG); and the metabolite is at least one selected from the group consisting 2-aminoadipic acid or mannose, or arabinonic acid, myo-inositol, cAMP, fructose, asy-dimethylarginine, citric acid, quinolinic acid, cysteine, spermidine, carnitine, isobutyrylcarnitine (C4), 3-methylhistidine, propionylcarnitine, and isocitric acid.
[15] The diagnosing method according to any one of the [2] to [9] above, wherein the glaucoma is pseudoexfoliative glaucoma/pseudoexfoliative syndrome (PEG); and the metabolite is selected from the group consisting acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formylkynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.
[16] The diagnosing method according to any one of the [1] to [15] above, wherein the diagnosis is to discriminate among the occurrence of retinal ganglion cell degeneration or the preceding susceptibility of retinal ganglion cells to degeneration stress or cell death, or the vulnerability of retinal ganglion cells.
[17] The diagnosing method according to any one of the [1] to [16] above, comprising a step of referring to the result of comprehensive proteome analysis in the molecular profiles of proteinaceous molecules contained in extracellular vesicle (EVs) in the aqueous humors.
[18] The diagnosing method according to any one of the [1] to [17] above, comprising a step of referring to findings in proteome analysis and/or gene expression analysis using clinical specimens.
[19] The diagnosing method according to any one of the [1] to [18] above, comprising a step of referring to information pertaining to factors in tear fluid, blood, and/or other body fluids.
[20] A diagnosing marker for evaluating the risk of ocular disease development or for diagnosing the disease progression of end-stage ocular diseases, which is used in the diagnosing method according to any one of the [1] to [19] above.
[21] The diagnosing marker according to the [20] above, which is at least one selected from the group consisting of sugar or polyol, a product in the citric acid cycle, acylcarnitine, polyamine, amino acid, and cAMP.
[22] The diagnosing marker according to the [21] above, wherein the sugar or polyol is arabinonic acid, myo-inositol, or fructose; the product in the citric acid cycle is citric acid or isocitric acid; the acylcarnitine is carnitine, isobutyrylcarnitine (C4), or propionylcarnitine; the polyamine is spermidine; and the amino acid is asy-dimethylarginine, quinolinic acid, cysteine, or 3-methylhistidine.
[23] The diagnosing marker according to the [21] or [22] above, wherein the sugar or polyol is arabinonic acid or myo-inositol; the acylcarnitine is isobutyrylcarnitine (C4); and the amino acid is cysteine.
[24] The diagnosing marker according to the [20] above, which is at least one selected from the group consisting of 2-aminoadipic acid, mannose, arabinonic acid, myo-inositol, cAMP, fructose, asy-dimethylarginine, citric acid, quinolinic acid, cysteine, spermidine, carnitine, isobutyrylcarnitine (C4), 3-methylhistidine, propionylcarnitine, GSH (glutathione), alanine, spermine, asparagine, choline, glutamine, glutamic acid, pyroglutamic acid, acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formylkynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.
[25] A diagnosing system for the risk of an ocular disease development or for the disease progression of an end-stage ocular disease comprising a means for collecting aqueous humor specimens and a means for analyzing the specimens by mass spectrometry (MS), which is used in the diagnosing method according to any one of the [1] to [19] above.
[26] The diagnosing system according to the [25] above, wherein the ocular disease is glaucoma.
[27] The diagnosing system according to the [25] above, wherein the ocular disease is corneal endothelial failures.

### Effect of the Invention

According to the present invention, the risk of glaucoma development can be diagnosed even in the pre-disease stage, for example, in the “very early stage with no clinically abnormal signs of ocular disease”. It is also possible to diagnose the susceptibility of retinal ganglion cells to degeneration or cell death, i.e., the vulnerability of retinal ganglion cells or the progression of end-stage glaucoma in the “visual impairment stage”.

It is very significant to provide a pathological stratification technique for bullous keratopathy, an intractable disease, by subjecting miRNAs, exosomes, SASP-related cytokines and metabolites, which are present in the ocular anterior tissue microenvironment, as biomarkers to a molecular diagnosis which is a fine and reproducible testing technique mutually interacting

The technology of the present invention, including a method of tracing glaucomatous disease-specific changes with the elimination of molecular species that leak into the aqueous humors due to nonspecific inflammation and the like, and a method of using, in the diagnosis of disease pathogenesis, proteinaceous molecules, miRNAs, metabolites, and exosome-containing molecules as molecules involved in network functioning in the ocular anterior microenvironment, and as assemblies of mutually interacting molecular species taking into comprehensive interpretation and using biostatistical methods such as Lasso analysis, is widely applicable not only to the diagnosis of glaucoma risk factors in the pre-disease stages of the disease or the determination of the progression of glaucoma at the “visual impairment stage” close to blindness, but also to diseases derived from the disruption of the network in the ocular anterior tissue microenvironment, thus opening great prospects for future medicine.

### BRIEF DESCRIPTION OF THE DRAWING

FIG. 1 comprehensively shows with radar charts the measurement results of levels of 18 proteinaceous cytokine molecules in aqueous humors collected prior to surgery from 77 patients including those who were scheduled for corneal transplant surgery, i.e., those with corneal endothelium failures in a single layer constituting corneal endothelial tissue exposed to the aqueous humors. It is shown that there is a great diversity in the profiles of functional cytokine molecules in the aqueous humors from patient to patient. It is suggested that this variation may lead to significant differences in graft survival. Necessity for testing the molecular conditions of the ocular anterior tissue microenvironment is shown.

FIG. 2 shows a schematic diagram of the molecular dynamics of the ocular anterior tissue microenvironment, in other words, the interaction among proteinaceous molecules such as cytokines, epigenetic factors such as miRNAs either contained or not contained in exosomes, and further metabolites and the like in the same microenvironment. Tissue homeostasis is maintained by the formation of a network that enhances or suppresses the expression and function of molecular species in a single molecular group, and the disruption of this network is thought to lead to the appearance of pathological states such as endothelial failures and glaucoma. The ocular anterior tissue microenvironment relates to the causes and consequences of endothelial cell degeneration and optic nerve cell degeneration, and constitutes interactions such as exacerbation, mitigation, and additive-synergistic-counteracting action.

FIG. 3 shows the diversity and quantitative differences in miRNAs in the aqueous humors of patients with corneal endothelial failures and cataract. This figure shows miRNA molecular species that are reduced by more than 2-fold with a significant difference as compared to cataracts. In general, miRNAs are known to suppress gene expressions, and hence their reduction indicates that many gene expressions are elicited in patients with corneal endothelial failures. This leads to fluctuations in the types and profiles of cytokines and metabolites. This is an example of the network shown in FIG. 2.

FIG. 4 shows the procedure for determining which molecular species that fluctuate in the aqueous humors are involved or not out of the miRNAs which are involved in SASP production that induces cellular senescence by proteinaceous cytokines from the examples shown in FIG. 3. The number of specimens is shown in the figure, and the kinetic analysis of approximately 3000 microRNA molecular species and 27 cytokine species was performed, and then 12 species of aqueous humors with large increases in SASP levels compared to controls are selected, and among them, the miRNAs with a concentration commonly decreased in aqueous humors are selected.

FIG. 5 shows in highlight the five miRNAs selected by the method shown in FIG. 4. It shows the possibility that the miRNAs that cause epigenetic gene expression changes in the surrounding tissues and cells in the aqueous humors, which is the microenvironment of the ocular anterior tissues, rather than the proper names of molecular species, are indeed selectively fluctuated and may act to disrupt tissue homeostasis.

FIG. 6 shows a flowchart illustrating the pathway of the effect of molecular dynamics in the aqueous humors on intraocular pressure elevation or visual field impairment.

FIG. 7 shows a cluster analysis, performed after trabeculotomy known as glaucoma surgery, on various aqueous humors from disease states that exhibit bullous keratopathy and inflammatory and non-inflammatory pathologies. It is also called as a heat map. In each map shown as a table, patients are shown on the vertical axis, and the types of cytokines tested are shown on the horizontal axis. It can be found that cytokine concentration profiles in the aqueous humors and the clusters formed are different among the four disease states. It is shown that, like miRNAs, cytokines are important molecular species that should be tested in the aqueous humors in relation to the disease state.

FIG. 8 shows the result of analyzing what cytokines have a possibility to be involved in differences in disease pathogenesis by measuring the levels in the aqueous humors for each causative disease of corneal transplantation, and then performing principal component analysis (PCA) separately for bullous keratopathy, Fuchs' corneal endothelial dystrophy, post-glaucoma surgery, and corneal leukoma/keratoconus. This study demonstrated that MCP-1, IP-10, IL-6, IL-8, IL-1ra, Eotaxin, IL-7, etc. are highly involved in pathological divergence. This is one of the scientific validations of the involvement of molecular dynamics of aqueous humors in the pathogenesis of corneal endothelium failures.

FIG. 9 shows a principal component analysis similar to that of FIG. 7 in four separate disease states the same as those in the cluster analysis of FIG. 6. This study demonstrated that MCP-1, IP-10, Eotaxin, IL-6, IL-8, IL-1Ra, IL-7, G-CSF, and GM-CSF are highly involved in pathological divergence. This is one of the scientific validations of the involvement of molecular dynamics of aqueous humors in the pathogenesis of corneal endothelium failures.

FIG. 10 shows a test of the association between the prognosis of corneal transplantation and the aqueous humor cytokine profile. This is the first finding to confirm that cytokines in the aqueous humors do indeed influence graft survival after corneal transplantation by a principal component analysis. This is the demonstration that graft survival is suppressed by the functional degeneration of the graft through its to the aqueous humors, where proteinaceous cytokine molecules constitute the ocular anterior tissue microenvironment, as described previously. The three molecules of IFNγ, IL-17, and PDGFββ were demonstrated to be the causative molecular species for graft failures.

FIG. 11 shows the commonality in the reduction of miRNA34a-5p and miRNA378a-3p between corneal endothelium pathological tissues and cultured human corneal endothelial cells with cell state transition. This indicates that molecular dynamics in the aqueous humors is involved in the degeneration and pathogenesis of ocular anterior-environment-related tissues, including glaucoma. This suggested a possibility that the same miRNAs, miRNA34a-5p and miRNA378a-3p, may be reduced in both the pathological tissues and the cultured cells with cell state transition, inducing epigenetic gene expression changes and then inducing changes in expressions of proteinaceous molecules, metabolites and metabolic enzymes related to metabolite dynamics.

FIG. 12 shows a conceptual diagram based on the idea of FIG. 10 as a model. The idea is that the decreased expressions of miR34a-5p and 378a-3p in endothelial cells induce changes in the products of such cells, and the products thus changed amplify the changes of neighboring cells in the form of cell competition or cell synchronization, leading to irreversible disease pathogenesis.

FIG. 13 shows the experimental results verifying the ideas of FIGS. 10 and 11. That is, it reveals that endothelial cells with reduced expressions of miRNA34a-5p and miRNA378a-3p may induce senescence and degeneration in the neighboring cells by extracellularly releasing large amounts of IL-8, MCP1, and VEGF, which are SASPs.

FIG. 14 shows that it was confirmed that exosomes released are different depending on cell types. It is a study about the diversity of exosomes which contain miRNAs, proteins, lipids, etc., and have a significant impact on the function of the neighboring cells. It was confirmed that the amounts of proteins corresponding to exosomes released from cells which are different in their expression levels of miR34a-5p and miRNA378a-3p were hardly variable such that they were 220 ng, 240 ng, 200 ng, and 260 ng. On the other hand, there were significant differences in the types of released exosomes identified with exosome surface markers, with lower expression of intracellular miRNA34a-5p and miRNA378a-3p leading to increased production of exosomes with CD9, CD63 and CD81 surface markers. It was demonstrated that the exosomes released by from cells with cell state transition, such as senescent cells and transformed cells, with reduced expression of miRNA34a-5p and miRNA378a-3p differ from cells without cell state transition in that they are CD9 and CD63 double-positivity.

FIG. 15, also relating to FIG. 13, shows the verification, by the difference in the types of miRNAs contained in exosomes, that if the exosomes released from cells are different depending on the cells, then the functional molecules contained in the exosomes will also be different. It is clear that the four types of miRNAs shown in the figure increase in amount as the cells degenerate. A possibility is indicated that such phenomenon occurs in the corneal endothelial tissue that constitutes the ocular anterior tissue microenvironment, leading to functional degeneration of the surrounding tissues (including glaucoma-related tissues) mediated by exosomes in the aqueous humors.

FIG. 16 shows a schematic diagram of the example shown in the previous figure. It shows that these molecules are responsible for the organization of intra-tissue network that causes large fluctuations in molecular dynamics of aqueous humors.

FIG. 17 shows the overall picture of the approach taken in the present invention in the search for markers of glaucoma development and progression in the aqueous humors, which constitutes the other part of the present invention. As of yet, there are no markers for the development of glaucoma in the pre-disease stage or objective methods for determining the progression of mid- to late-stage glaucoma available in the clinical field. The present invention attempts to solve this unmet problem within the several stages from healthy eye to blindness by making full use of MS-based testing of metabolites in the aqueous humors.

FIG. 18 shows a volcano plot representing the results of LC-MS measurements of metabolites in the glaucoma group with the cataract group as a control. The vertical axis represents −log₁₀ (p-value), and the horizontal axis represents log₂ (FC). Light-colored and dark-colored dots in the figure indicate amino acid metabolites and other metabolites, respectively. Note that the p-value means the statistically significant difference, and the FC means the ratio of the mean value of the glaucoma group to the mean value of the cataract group (Fold Change).

FIG. 19 shows a volcano plot representing the results of LC-MS measurements of metabolites in the corneal transplant group with the cataract group as a control. The vertical axis represents −log₁₀ (p-value), and the horizontal axis represents log₂ (FC). Light-colored and dark-colored dots in the figure indicate amino acid metabolites and other metabolites, respectively.

FIG. 20 shows a volcano plot representing the results of LC-MS measurements of metabolites in the cell injection group with the cataract group as a control. The vertical axis represents −log₁₀ (p-value), and the horizontal axis represents log₂ (FC). Light-colored and dark-colored dots in the figure indicate amino acid metabolites and other metabolites, respectively.

FIG. 21 shows a volcano plot representing the results of GC-MS measurements of metabolites in the glaucoma group with the cataract group as a control. The vertical axis represents −log₁₀ (p-value), and the horizontal axis represents log₂ (FC). Light-colored and dark-colored dots in the figure indicate amino acid metabolites and other metabolites, respectively.

FIG. 22 shows a volcano plot representing the results of GC-MS measurements of metabolites in the corneal transplant group with the cataract group as a control. The vertical axis represents −log₁₀ (p-value), and the horizontal axis represents log₂ (FC). Light-colored and dark-colored dots in the figure indicate amino acid metabolites and other metabolites, respectively.

FIG. 23 shows a volcano plot representing the results of GC-MS measurements of metabolites in the cell injection group with the cataract group as a control. The vertical axis represents −log₁₀ (p-value), and the horizontal axis represents log₂ (FC). Light-colored and dark-colored dots in the figure indicate amino acid metabolites and other metabolites, respectively.

FIG. 24 shows a heat map analysis based on the combined data of LC-MS and GC-MS of preoperative aqueous humors from cell injection regenerative medicine patients and corneal transplant patients as representatives of corneal endothelial failures, and of aqueous humors from three glaucoma types (POAG, NTG, PEG), and cataract patients as controls. The vertical axis represents the disease types and the horizontal axis represents the metabolites. On the right is a principal component analysis (PCA) of the results in the aqueous humors from the three glaucoma types and cataract patients as controls. The metabolites were found to be distinctly different depending on the pathological condition, with polyamines and carnitine contributing to the pathological divergence in glaucoma as opposed to cataract.

FIG. 25 shows a venn diagram representing the differences in metabolites that vary among patients of cell injection, preoperative patients of corneal transplantation, and glaucomatous disease PEGs. The results of both LC-MS and GC-MS are combined.

FIG. 26 shows a venn diagram representing the differences in metabolites that vary among patients of cell injection, preoperative patients of corneal transplantation, and glaucomatous disease PEGs. It is the results of LC-MS.

FIG. 27 shows a venn diagram representing the differences in metabolites that vary among patients of cell injection, preoperative patients of corneal transplantation, and glaucomatous disease PEGs. It is the results of GC-MS.

FIG. 28 shows a venn diagram representing the differences in metabolites that vary among patients of cell injection, preoperative patients of corneal transplantation, and glaucomatous disease NTG. The results of both LC-MS and GC-MS are combined.

FIG. 29 shows a venn diagram representing the differences in metabolites that vary among patients of cell injection, preoperative patients of corneal transplantation, and glaucomatous disease POAG. The results of both LC-MS and GC-MS are combined.

FIG. 30 shows a venn diagram representing the relationship between the number of metabolites that vary in the three glaucoma types (NTG, PEG and POAG), and a list of metabolites that vary in common among the three types of the disease.

FIG. 31 shows a set of models (combinations of metabolites and their weights) extracted by selecting metabolites that contribute to disease discrimination from all metabolites using LASSO (least absolute shrinkage and selection operator), one of machine learning methods: the results for the three glaucoma disease types combined vs. cataract.

FIG. 32 shows a set of models (combinations of metabolites and their weights) extracted by selecting metabolites that contribute to disease discrimination from all metabolites using LASSO: PEG vs. cataract.

FIG. 33 shows a set of models (combinations of metabolites and their weights) extracted by selecting metabolites that contribute to disease discrimination from all metabolites using LASSO: POAG vs. cataract.

FIG. 34 shows a set of models (combinations of metabolites and their weights) extracted by selecting metabolites that contribute to disease discrimination from all metabolites using LASSO: NTG vs. cataract.

FIG. 35 shows a set of models (combinations of metabolites and their weights) extracted by selecting metabolites that contribute to disease discrimination from all metabolites using LASSO: the results for the three glaucoma disease types combined vs. cataract.

FIG. 36 shows a set of models (combinations of metabolites and their weights) extracted by selecting metabolites that contribute to disease discrimination from all metabolites using LASSO: PEG vs. cataract.

FIG. 37 shows a set of models (combinations of metabolites and their weights) extracted by selecting metabolites that contribute to disease discrimination from all metabolites using LASSO: POAG vs. cataract.

FIG. 38 shows a set of models (combinations of metabolites and their weights) extracted by selecting metabolites that contribute to disease discrimination from all metabolites using LASSO: NTG vs. cataract.

FIG. 39 shows the results of the determination of arabinonic acid amount in the aqueous humors for a corneal endothelial failure group, a cataract group, and a glaucoma group by mass spectrometry using LC-MS, in the upper figure. The vertical axis shows the concentration of arabinonic acid (relative amount to the standard material). The lower figure shows the ROC curve for glaucoma evaluation when arabinonic acid is used as a marker. The vertical and horizontal axes indicate the true positive rate (TPR) and false positive rate (FPR), respectively.

FIG. 40 shows the results of the determination of myo-inositol amount in the aqueous humors for a corneal endothelial failure group, a cataract group, and a glaucoma group by mass spectrometry using GC-MS, in the upper figure. The vertical axis shows the concentration of myo-inositol (relative amount to the standard material). The lower figure shows the ROC curve for glaucoma evaluation when myo-inositol is used as a marker. The vertical and horizontal axes indicate TPR and FPR, respectively.

FIG. 41 shows the results of the determination of cAMP amount in the aqueous humors for a corneal endothelial failure group, a cataract group, and a glaucoma group by mass spectrometry using LC-MS, in the upper figure. The vertical axis shows the concentration of cAMP (relative amount to the standard material). The lower figure shows the ROC curve for glaucoma evaluation when cAMP is used as a marker. The vertical and horizontal axes indicate TPR and FPR, respectively.

FIG. 42 shows the results of the determination of fructose amount in the aqueous humors for a corneal endothelial failure group, a cataract group, and a glaucoma group by mass spectrometry using GC-MS, in the upper figure. The vertical axis shows the concentration of fructose (relative amount to the standard material). The lower figure shows the ROC curve for glaucoma evaluation when fructose is used as a marker. The vertical and horizontal axes indicate TPR and FPR, respectively.

FIG. 43 shows ROC curves representing the results of the evaluation of marker performance for the five metabolites arabinonic acid, cAMP, cysteine, spermidine, and isobutyrylcarnitine (C4), with cataract as negative and all glaucoma, regardless of cause, as positive. The vertical and horizontal axes represent TPR and FPR, respectively.

FIG. 44 shows a color map representing Pearson product-moment correlation coefficients between metabolites for the 15 metabolites that vary in common among the three glaucoma disease types.

FIG. 45 shows ROC curves representing the results of the evaluation of marker performance for the five metabolites myo-inositol, cAMP, cysteine, spermidine, and isobutyricarnitine (C4) with cataract as negative and all glaucoma, regardless of cause, as positive. The vertical and horizontal axes represent TPR and FPR, respectively.

FIG. 46 shows a procedural diagram of a biostatistical method for extracting metabolites correlated with miRNAs involved in the pathogenesis of corneal endothelial failure and the family members thereof. The metabolites correlated with miRNAs involved in the pathogenesis of corneal endothelial failure and the family members thereof were extracted through calculating regression coefficients from Lasso using 2565 miRNAs as explanatory variables and 212 metabolites detected by mass spectrometry in the aqueous humors extracted from patients (n=29) with corneal endothelial failures (bullous keratopathy) as objective variables.

FIG. 47 shows metabolites that fluctuate in correlation with miRNAs which are detected in the aqueous humors and fluctuate with corneal endothelia; cell degeneration. The followings were extracted: (1) Metabolites correlated with SASP (Senescence-associated secretory phenotype)-suppressing miRNAs in the aqueous humors; (2) metabolites correlated with miRNA highly expressed in the culture supernatant of degenerated cultured corneal endothelial cells; and (3) metabolites correlated with miRNAs expressed at the reduced levels in the same degenerated cells.

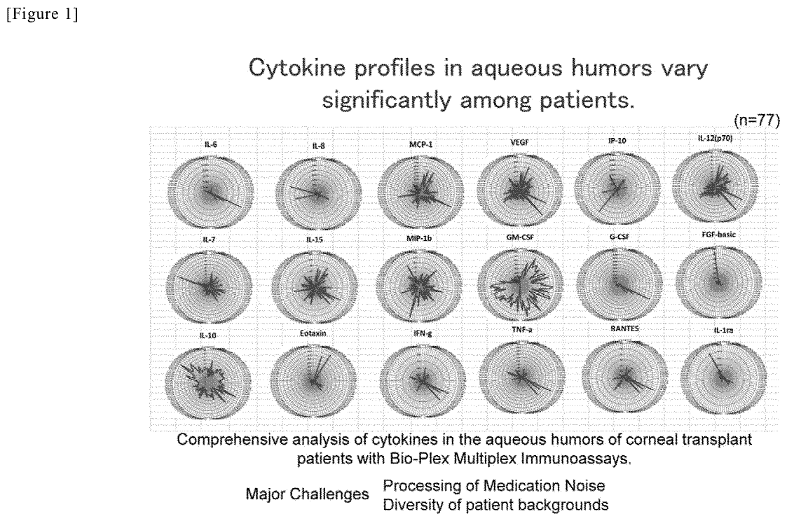

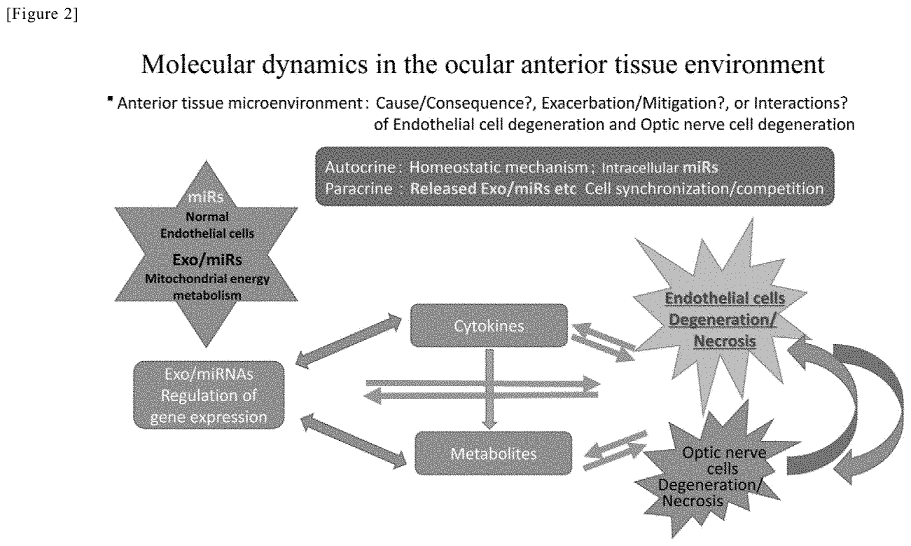

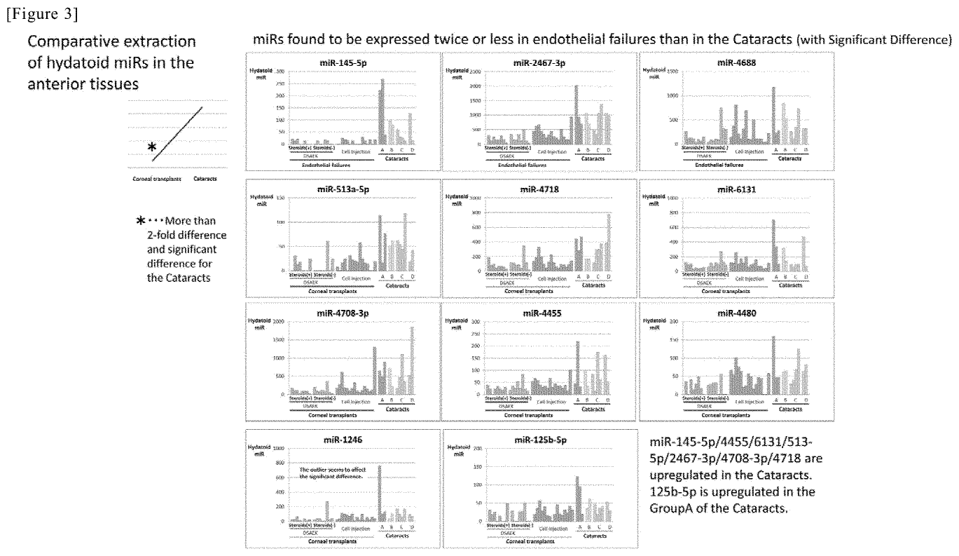

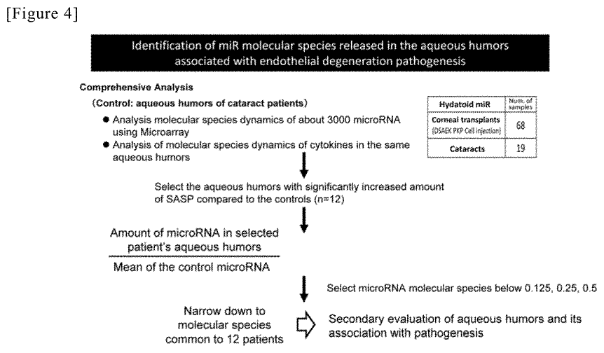

### EMBODIMENTS FOR CARRYING OUT THE INVENTION

The present invention will be described in detail below.

### 1. Diagnosis Method and Diagnosis Marker According to the Present Invention

The diagnosis method of the present invention (hereinafter referred to as the “diagnosis method of the present invention”) is a method for diagnosing the risk of developing an ocular disease or for diagnosing the pathogenic progression of an end-stage ocular disease by molecular dynamics analysis of the ocular anterior tissue microenvironment, and characterized by comprising a step of measuring the concentration level of a metabolite appearing in the collected aqueous humors by mass spectrometry (MS).

In addition, the diagnosis marker according to the present invention (hereinafter referred to as the “diagnosis marker of the present invention”) is a marker for evaluating the risk of developing ocular diseases or for diagnosing the pathogenic progression of end-stage ocular diseases, and is used in the diagnosis method of the present invention.

The diagnosis method of the present invention is a method that can be used to practically diagnose the risk of developing and the advanced pathogenesis of ocular diseases related to the disruption of the ocular anterior tissue microenvironment, characterized by comprehensive molecular dynamics analysis technology, which can only be possible by measuring the concentration levels of metabolites as well as testing, in each disease state, the molecular profiles of proteinaceous molecules, nucleic acids, and extracellular vesicles that appear in the ocular anterior tissue microenvironment, especially in the aqueous humors, for ocular diseases caused by disruption of the ocular anterior tissue microenvironment, particularly corneal endothelial failures including bullous keratopathy and Fuchs' corneal dystrophy, as well as pathologically related diseases commonly referred to as glaucoma. The method includes a comprehensive molecular kinetic analysis technique that measures the concentration levels of metabolites in addition to testing, in each disease state, the molecular profiles of proteinaceous molecules, nucleic acid-based molecules, and extracellular vesicles that appear in the ocular anterior tissue microenvironment, particularly in sampled aqueous humors.

As shown in Test Examples described later, concentration levels of proteinaceous molecules, nucleic acids, extracellular vesicles, and metabolites that appear in aqueous humors collected from patients eligible to corneal transplantation, patients eligible to a regenerative medicine with cultured human corneal endothelial cell injection, cataract patients, normal tension glaucoma (NTG) patients, primary open-angle glaucoma (POAG) patients, or pseudoexfoliative glaucoma (PEG) patients were determined by multiplex bead assays, enzyme-linked immunoassays (ELISA), miRNA chips, gas chromatography-mass spectrometry (GC-MS) or liquid chromatography-mass spectrometry (LC-MS) and compared to other diseases, with the cataract group as a control.

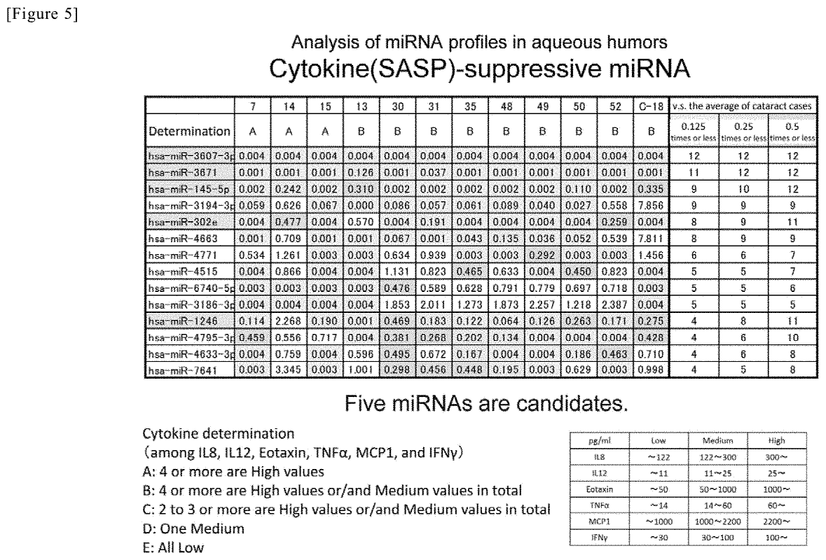

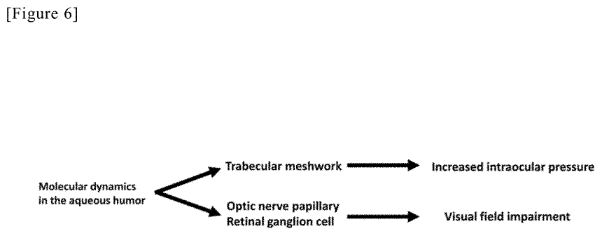

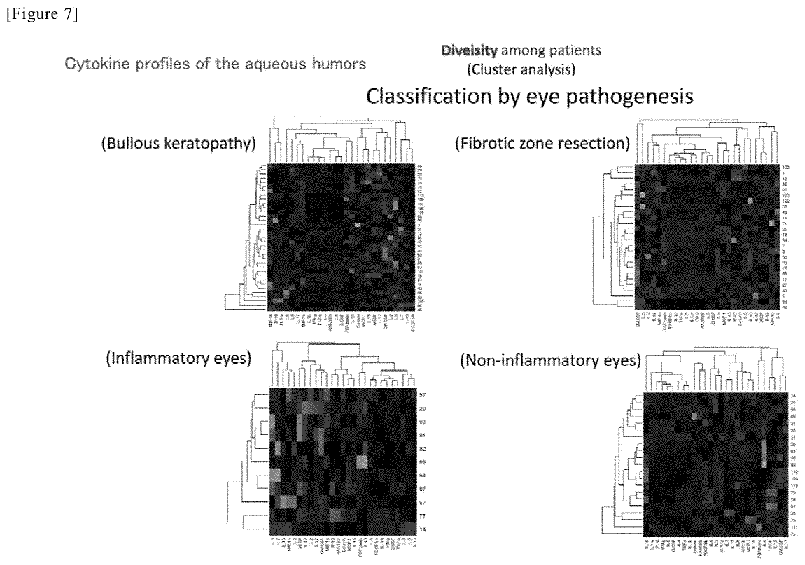

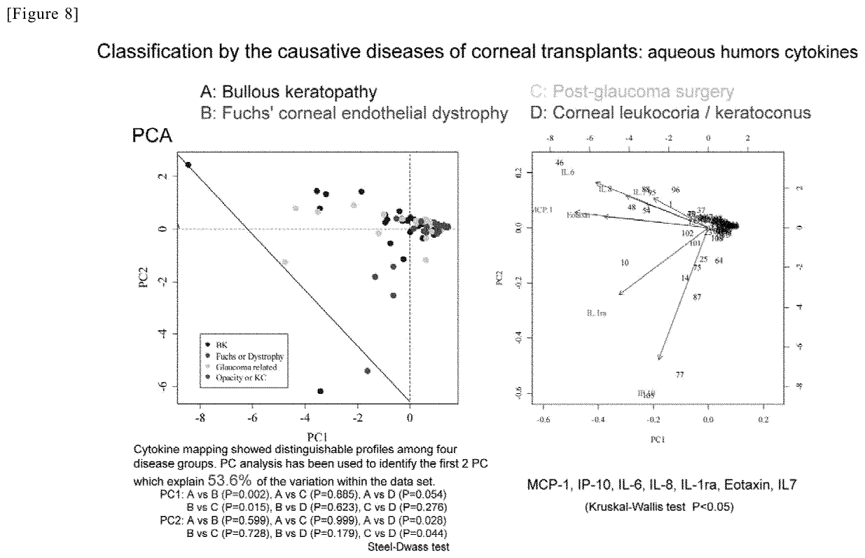

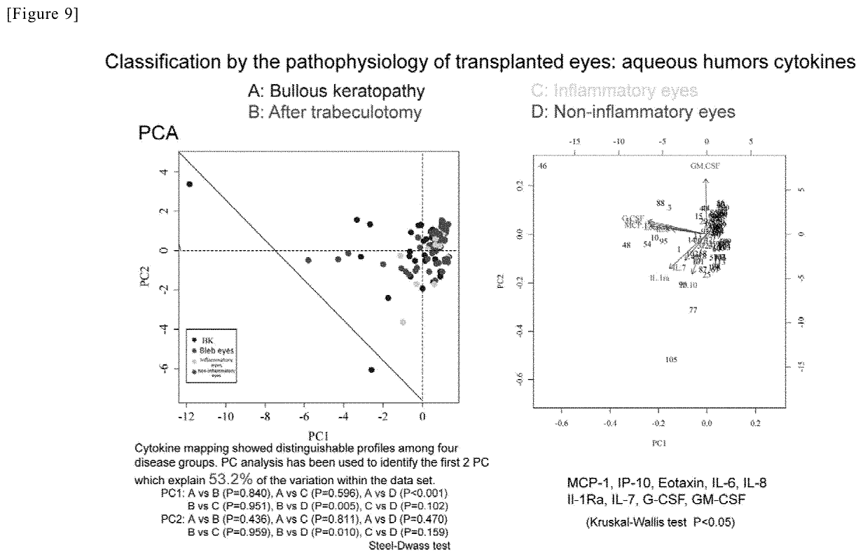

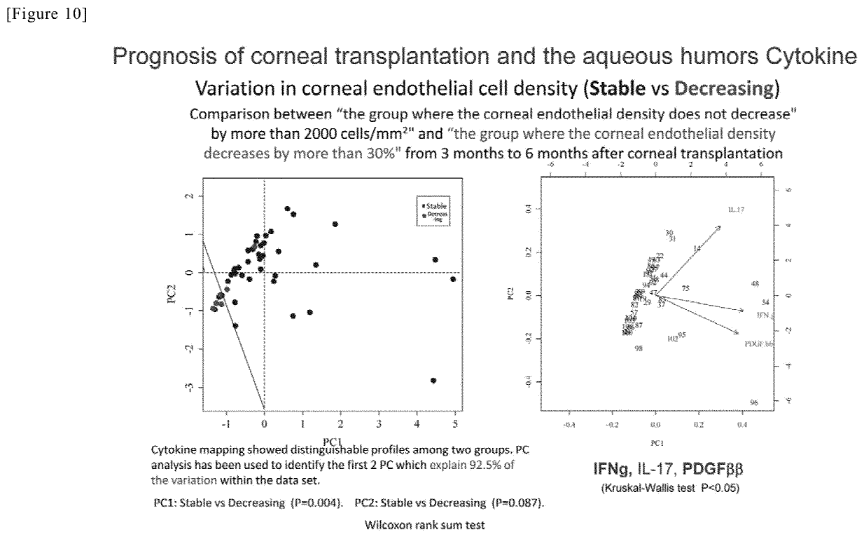

Tests for cytokines, one of the environmental factors in the aqueous humors of patients with bullous keratopathy, revealed that (1) there is a diversity of microenvironmental molecular profiles in the aqueous humors among bullous keratopathy patients, and that (2) the microenvironmental molecular profiles can be differentiated by the causative diseases of bullous keratopathy patients (FIGS. 7 to 9), and confirmed that (3) the diversity is not due to differences in gender or age. The tests observed that (4) three SASP-related cytokines are identified in the aqueous humors and that the levels of these three cytokines in aqueous humors correlate with the effect to maintain corneal endothelial cell density after corneal transplantation, namely that there is a biostatistically significant correlation of the three cytokines with in vivo effects. We were the first in the world to verify that the long-term prognosis of transplantation outcomes after corneal transplantation can be determined by testing preoperative molecular profiles in aqueous humors, i.e., that molecular species in the aqueous humors may be related to the impairment of cell function in the ocular anterior tissue microenvironment (FIG. 10). In addition, we followed the diversity in molecular profiles of the above aqueous humors in relation to long-term prognosis of corneal transplant patients and found a correlation of the long-term prognosis with five miRNA molecular species, succeeding to cytokines mentioned above (see FIG. 5). The results validated the utility of this technology as a standard technique for diagnosing the prognosis of corneal transplantation and cell injection regenerative medicine, i.e., the practical usefulness of the technology to distinguish the degenerated or non-degenerated corneal endothelial cells.

Bullous keratopathy, a typical disorder of corneal endothelial tissue failures, is known to cause a significant decrease in corneal endothelial cell density and a flattening and enlargement of the cell shape, and Fuchs' corneal endothelial dystrophy is also known to cause a formation of Guttata, in which extracellular matrix components (ECM) deposit from the endothelial cells to the posterior surface of Descemet's membrane. The molecular mechanism of why this reduction in cell density, cell enlargement, and Guttata formation occurs is unknown. Cultured human corneal endothelial cells also frequently undergo a cell state transition to cells with morphology different from that of small, cobblestone-like corneal endothelial cell monolayer, but the molecular mechanism of this cell state transition is also unknown.

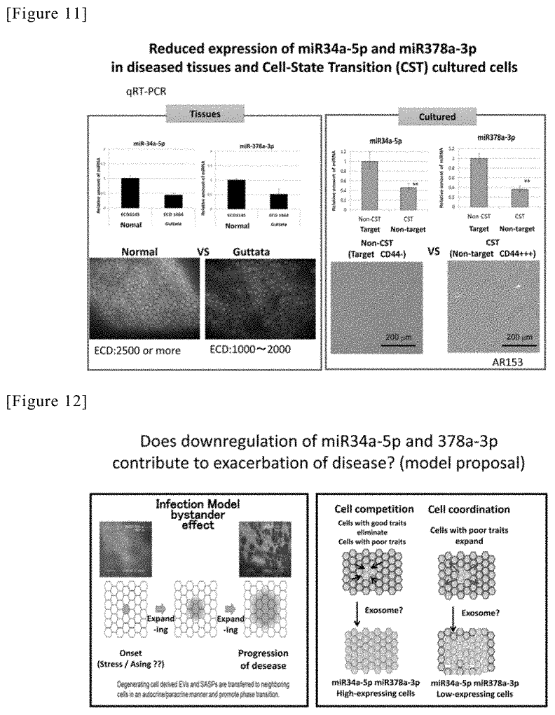

First, miRNAs that were down-regulated in both pathogenic tissues and cell state transitioned cultured cells were selected by comprehensive analysis of miRNAs, and miRNA34a-5p and miRNA378a-3p were selected by quantitative RT-PCR validation (FIG. 11).

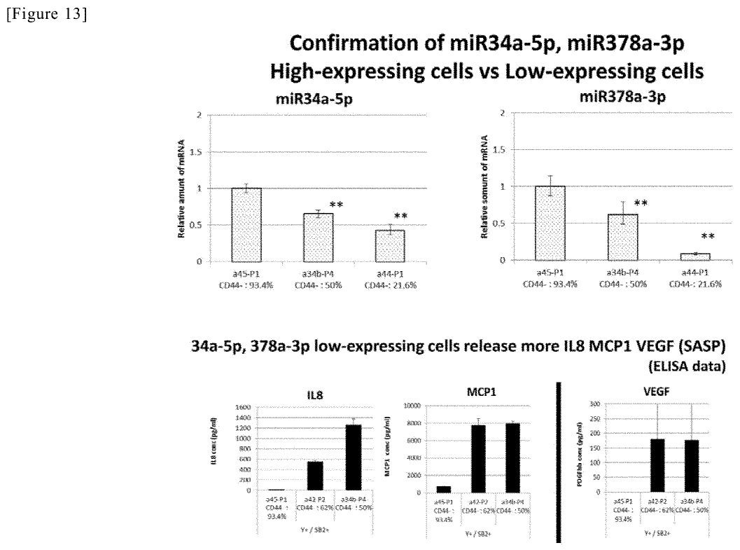

The present inventors hypothesized that cell competition and cell synchronization play important roles in disease progression, and proposed the idea that the paracrine action of exosomes, as shown in FIG. 12, may either aggravate or attenuate the disease progression. miRNA34a-5p and miRNA378a-3p were analyzed from the viewpoint that they might affect the quantity and quality of exosomes produced by cultured human corneal endothelial cells. It is known that SASP also plays a major role in cell competition and cell synchronization. It was verified by the Elisa method that the lower the expressions of intracellular miRNA34a-5p and miRNA378a-3p, the more secretion from the cells of IL8, MCP1, and VEGF, which are SASP-related cytokines (FIG. 13). The possibility that these may also induce senescence of the neighboring cells in a paracrine manner was verified.

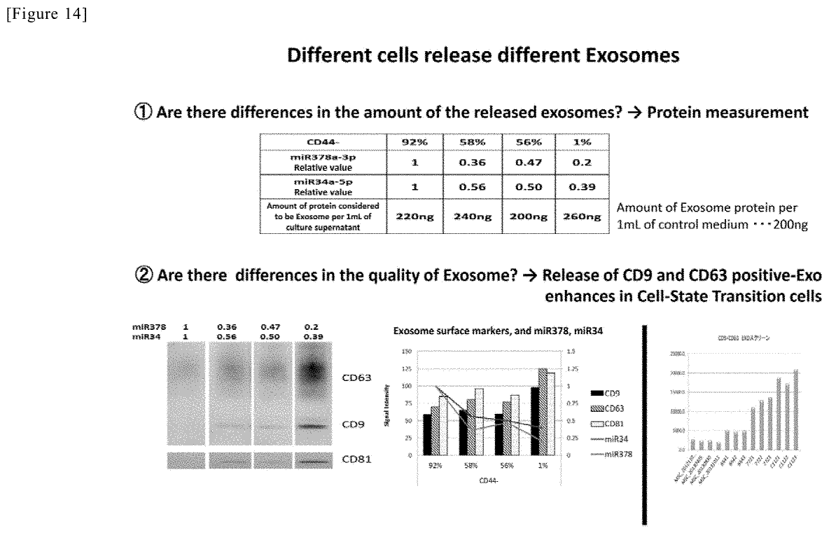

Next, we also examined the polarized profile of exosomes. The amounts of exosomes released by cells with distinct miRNA34a-5p and miRNA378a-3p expression levels were examined. The amounts of proteins corresponding to exosomes released by cells with distinct miRNA34a-5p and miRNA378a-3p expression levels were hardly variable such that they were 220 ng, 240 ng, 200 ng, and 260 ng. On the other hand, significant differences were observed in the types of released exosomes identified with exosome surface markers. The production of exosomes with CD9, CD63, and CD81 surface markers increases with reduced expression of intracellular miRNA34a-5p and miRNA378a-3p, and exosomes released by cell state-transitioned cells with reduced miRNA34a-5p and miRNA378a-3p are double positive for CD9 and CD63, unlike non-cell state-transitioned cells (FIG. 14).

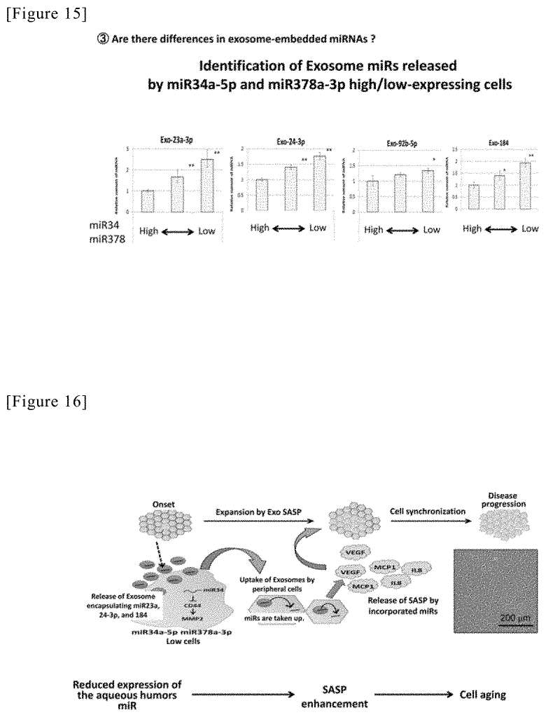

Furthermore, the lower the expression of miRNA34a-5p and miRNA378a-3p in the cells, miRNA23a, miRNA24, and miRNA184 were identified as miRNAs encapsulated in exosomes and released extracellularly (FIG. 15).

In order to investigate the most important point, namely, whether there is a possibility that cell competition and cell synchronization may regulate disease progression even in diseased tissues, comprehensive measurement of miRNAs and cytokines in the aqueous humors was carried out in about 68 samples of pre-surgical aqueous humors from corneal transplant recipients and 19 samples of aqueous humors from cataract patients served as controls. Aqueous humors with a large increase in SASP were selected, and compared the amounts of miRNAs therein with those in control cataract patients, and thereby selected 5 types of miRNAs which were reduced when SASP was increased in the aqueous humors (FIG. 5).

In summary, it was proven that there exists a vicious cycle for disease progression, in which the reduction in miRNA34a-5p and miRNA378a-3p enhances release of exosomes encapsulating miRNA23a, miRNA24, miRNA184, etc., which are taken up in a paracrine manner by neighboring cells and thereby SASP secretion is enhanced by the action of encapsulated miRNAs, and then the SASP causes senescence and degeneration of the neighboring cells. The possibility that miRNAs released from endothelial cells in the form of inclusion body of exosomes as one of the molecular dynamics of the ocular anterior tissue microenvironment, which is the skeleton of the present invention, may be involved in the disease pathogenesis, including glaucoma, which is the subject of the present invention, through disruption of the network among ocular anterior tissues was revealed for the first time. This is one of the key elements supporting the probability of the present invention (FIG. 16).

The present inventors have already reported that there is diversity in the metabolites produced by cultured human corneal endothelial cell subpopulations, and that the clinical efficacy in cell injection regenerative medicine varies in relation to the type of metabolite (Junji Hamuro, et al., IOVS, 2020; Vol. 61, No. 2: 1-12). We think that there exists the following cycle: gene expression induced by cytokines produced and secreted by transplanted cultured cells and soluble miRNAs contained in released exosomes, and their miRNAs→production induction→alteration of the molecular profiles of the aqueous humors by the induced cytokines and metabolites, affecting the clinical efficacy. This suggests that even in vivo, cytokines, miRNAs, and metabolites produced by corneal endothelial tissue may be more or less relevant to the pathogenesis of corneal endothelium failures and glaucoma. This indicates the usefulness of the technique to identify candidate biomarker molecules that exist specifically in the aqueous humors and may be involved in disease pathogenesis, namely corneal endothelial cell degeneration and optic nerve cell degeneration.

Candidate molecular species other than cytokines and soluble miRNAs were also selected from metabolites. This is an advanced analysis of metabolites using GC-MS and LC-MS. Abnormalities in polyamine metabolic systems have been observed in patients with pseudoexfoliative glaucoma, including Spermidine and Spermine from Ornithine, which is linked to the urea cycle and the polyamine system. It is also clear that molecular species common to those occurring in Parkinson's disease and detected in spinal fluid are present among the candidate molecular species.

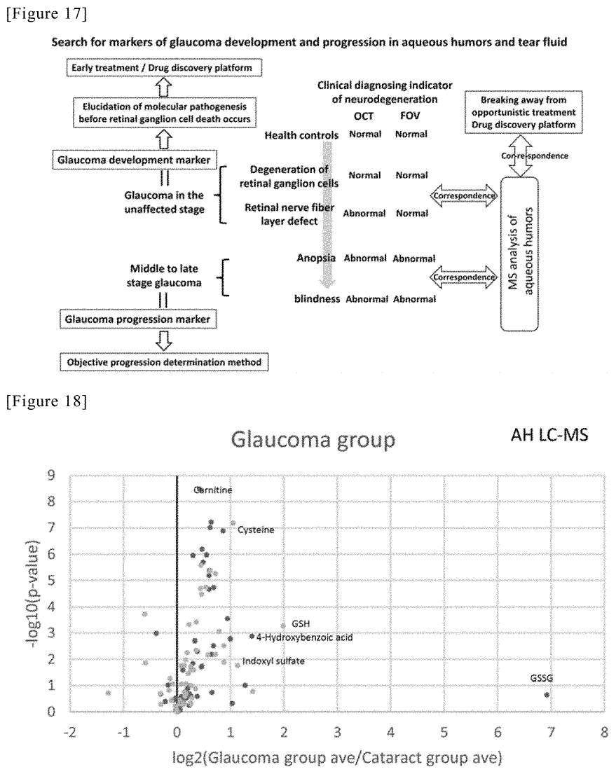

The procedure for the selection of markers in the aqueous humors for the present invention is shown in the figure (FIG. 17).

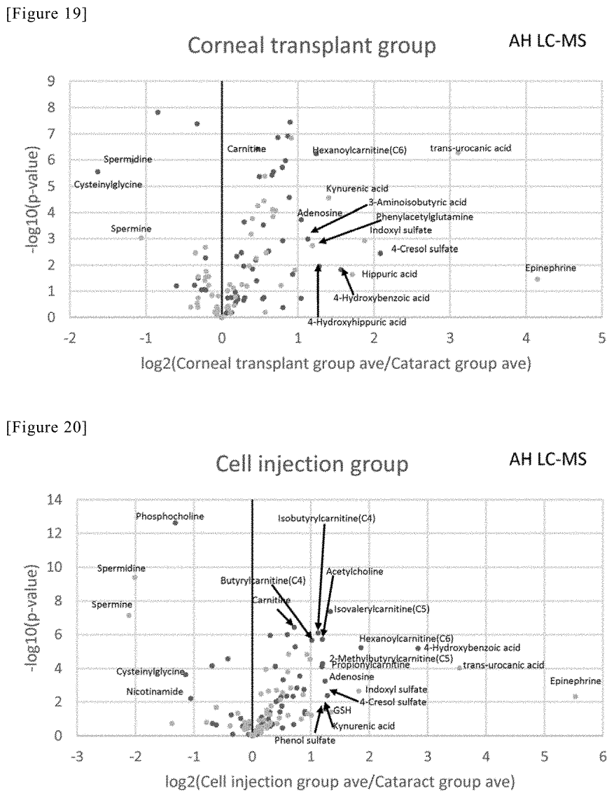

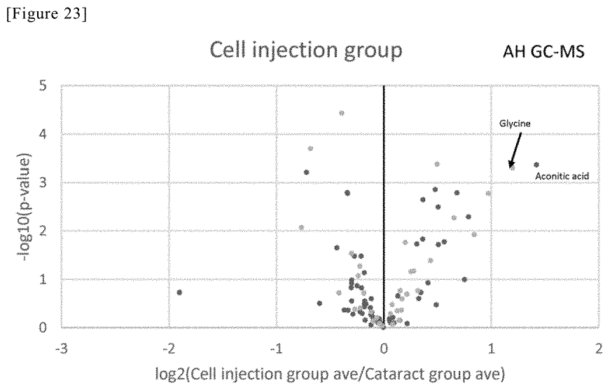

As shown in Test Examples described later, aqueous humor samples were collected from the following patients: cataract patients for the purpose of eliminating nonspecific molecular species in the aqueous humors; patients scheduled for corneal transplantation as the patients with another ocular anterior tissue dysfunction [patients with corneal endothelial failures] for the purpose of depicting intra-anterior chamber molecular species; and patients scheduled for cell injection regenerative medicine surgery. In addition, samples were also collected from glaucoma patients with different pathological types, such as normal tension glaucoma (NTG) patients, primary open-angle glaucoma (POAG) patients, or pseudoexfoliative glaucoma (PEG) patients. The concentration levels of metabolites appearing in the aqueous humors were measured by gas chromatography-mass spectrometry (GC-MS) or liquid chromatography-mass spectrometry (LC-MS). Using the cataract group as a control, the comparison of molecular profiles of the aqueous humors between groups by the Wilcoxon rank sum test was first performed for the patients scheduled for corneal transplantation, the patients scheduled for cell injection regenerative medicine surgery and each of the three disease-type distinct glaucoma groups (NTG, POAG, and PEG) to select molecular species specific to glaucoma disease. FIGS. 18 to 23 are Volcano Plots representing the results of measurements by LC-MS or GC-MS of metabolites in pre-surgery aqueous humors from patients in the glaucoma group, the corneal transplant group, and the cell injection group. The vertical axis shows −log₁₀ (p-value) and the horizontal axis shows log₂ (FC). In the figures, light-colored and dark-colored dots indicate amino acid metabolites and other metabolites, respectively. Note that the p-value means the statistically significant difference, and FC means the ratio (fold change) of the mean value of the glaucoma group to the mean value of the cataract group (FIGS. 18 to 23). Clear differences were found between cataract patients and the other three diseases.

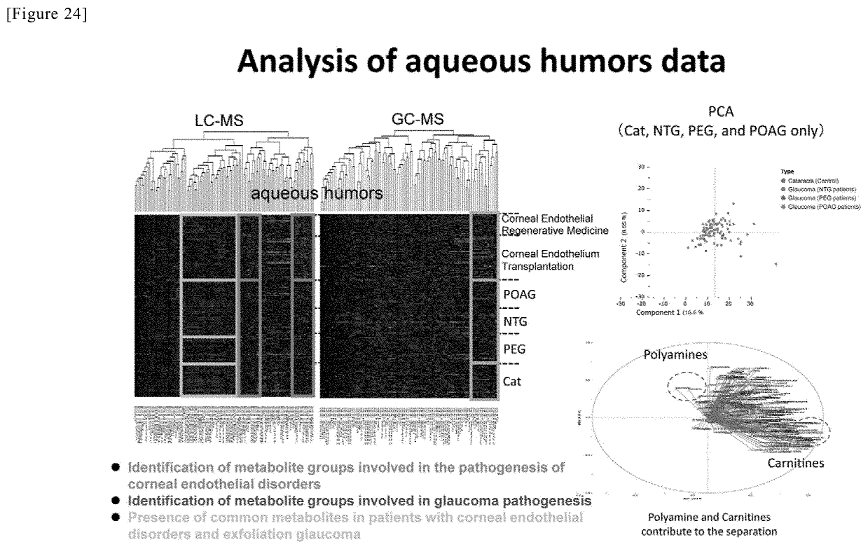

One of the major pillars of the present invention is to exclude not only the molecular species in the aqueous humors of cataract patients but also the molecular species appearing due to the corneal endothelial failures when selecting molecular species related to the pathogenesis of glaucoma in the aqueous humors. For that, we first performed the cluster analysis shown in FIG. 24 to see the differences in metabolites in the six diseases. On the right side of the same figure, shown is the result of principal component analysis that the metabolites in the aqueous humors of glaucoma patients are separated from those of cataract patients, which suggests that polyamine compounds and carnitine-related molecular species are involved in the divergence (FIG. 24).

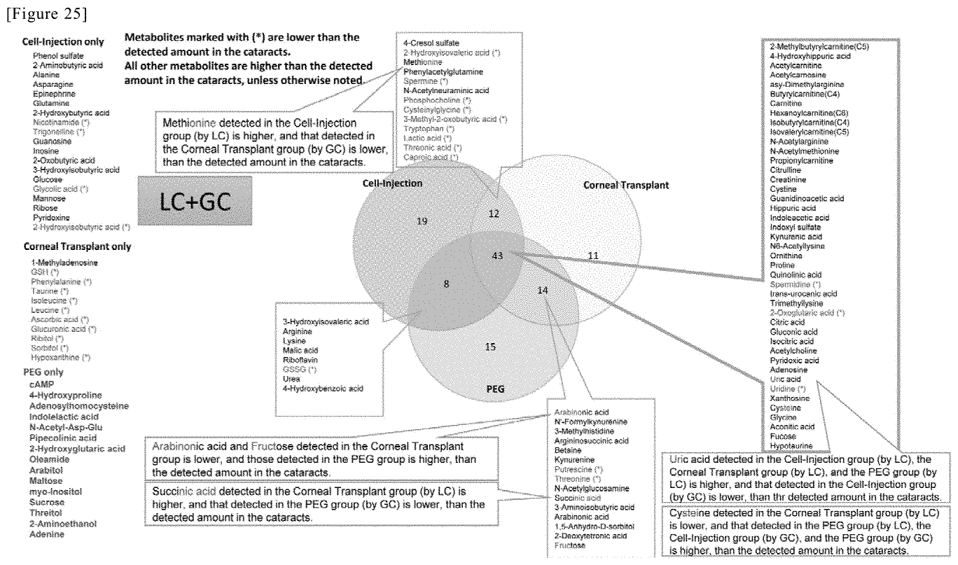

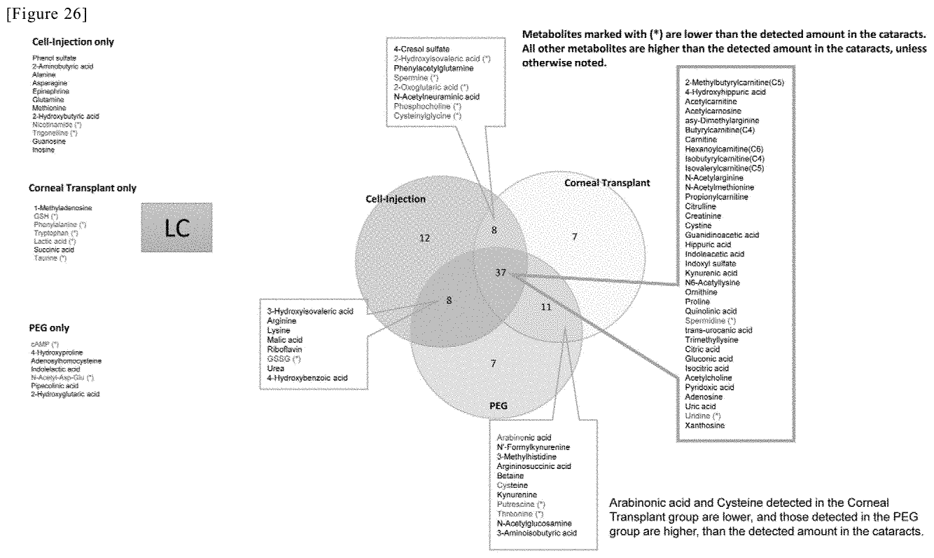

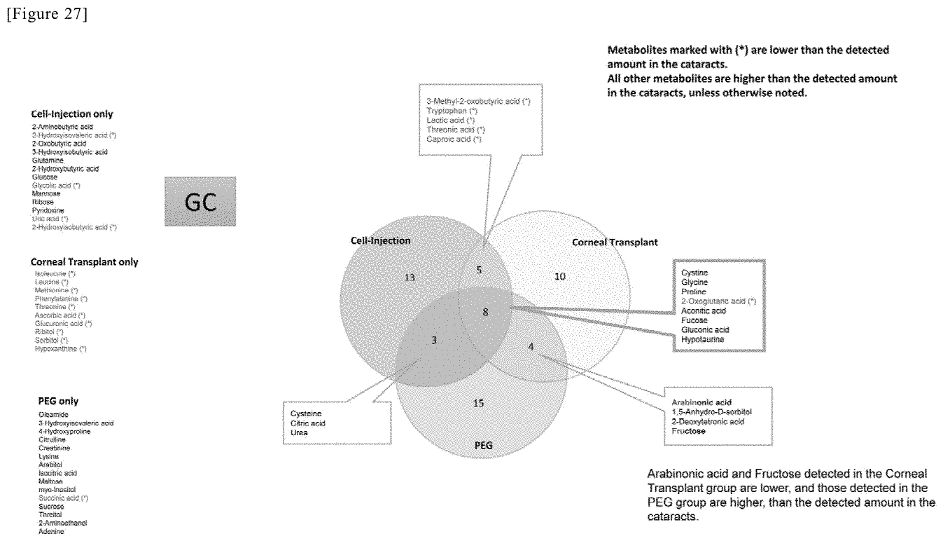

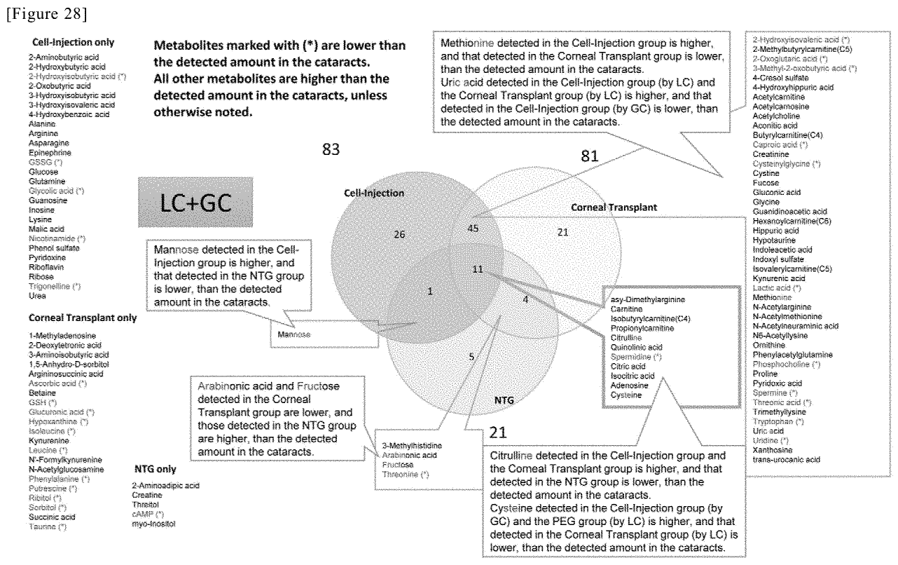

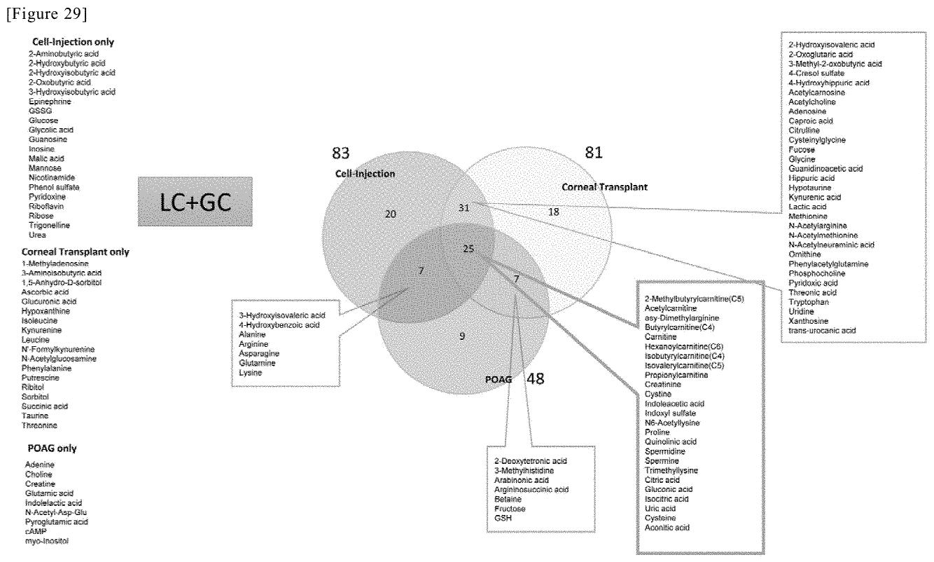

The metabolites include a variety of metabolites like those common to patients scheduled for corneal transplantation, patients scheduled for cell injection regenerative medicine surgery, and patients with glaucoma in each of the three disease groups with different pathologies, those present in each disease alone, and those common to two specific diseases among them. To exclude molecular species selectively present in the aqueous humors of patients with corneal endothelial failures, further Venn diagram analysis was performed as shown in FIGS. 25 to 29. Metabolites included specifically or in common were also described in the figures (a biostatistical analysis method called Cross Validation). The numbers in the Venn diagram indicate the number of metabolites. In this statistical analysis, only those showing an increase or decrease in comparison with cataract patients, with a p-value <0.05 indicating a significant difference were included in the analysis (FIGS. 25 to 29). In comparison with the pseudoexfoliative glaucoma group, metabolites detected by both LC-MS and GC-MS, as well as those detected by either alone, are described. Whereas, for those of NTG and POAG patients, only metabolites detected by both LC-MS and GC-MS are described.

As apparent from FIGS. 25, 28, and 29, 15 species out of 80 metabolites detected in the pseudoexfoliative glaucoma group are specific, 5 species out of 21 metabolites in total are specific in NTG, and 9 species out of 48 metabolites in total are specific in POAG. This demonstrates in part the usefulness of the disease-pathogenesis-specific metabolite selection method applied for the first time in the present invention.

If this method is used only for the three pathologic types of glaucoma, there are metabolites common to both the NTG group and the POAG group, metabolites common to both the NTG group and the PEG group, metabolites common to both the POGA group and the PEG group, and metabolites common to three of the NTG group, the POAG group and the PEG group.

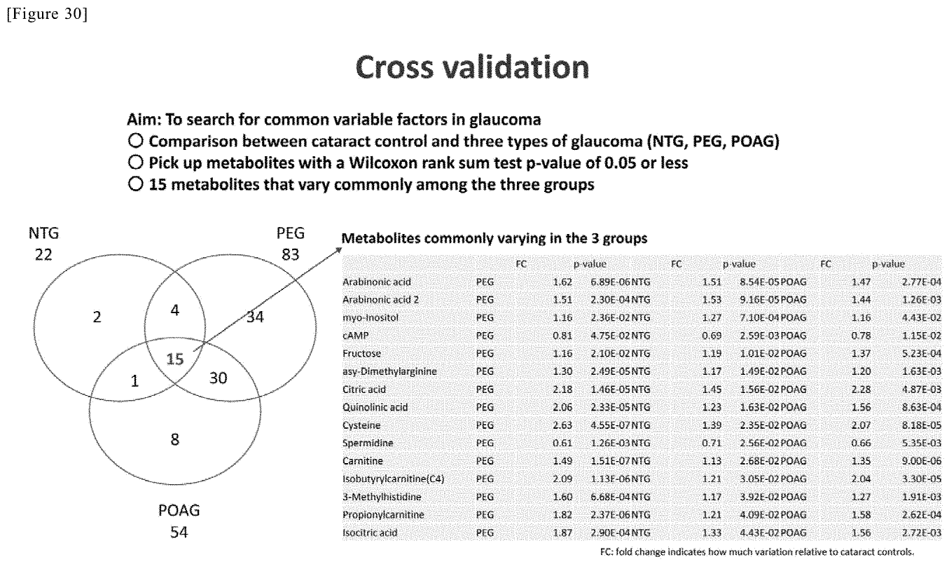

This relationship can also be represented by the Venn diagram like FIG. 30. Metabolites that vary in common among the three disease types are the 15 species in the center of the Venn diagram (maybe overlapped due to differences in measurement methods). Specifically, they are arabinonic acid, myo-inositol, cAMP, fructose, asy-dimethylarginine, citric acid, quinolinic acid, cysteine, spermidine, carnitine, is obutyrylcarnitine (C4), 3-methylhistidine, propionylcarnitine, and isocitrate.

These are considered to lie at a stage where it is difficult to determine the degree of progression by optical methods.

Consequently, statistically significant differences (p-value <0.05) were observed for 22 metabolites (maybe overlapped due to differences in measurement methods) in comparison with the NTG group, 54 metabolites (maybe overlapped due to differences in measurement methods) in comparison with the POAG group, and 83 metabolites (maybe overlapped due to differences in measurement methods) in comparison with the PEG group.

Among the metabolites that vary in common among the three disease types, myo-inositol, fructose, citric acid, cysteine, carnitine, and propionylcarnitine has been reported as molecules that are upregulated in the retinal tissue over the medium to long term in a rat model of optic neuropathy (Agudo-Barriuso, M., IOVS. 54, 4249-11 (2013)). The rat model of optic neuropathy used in the report does not provide any insight into the disease progression in human glaucoma patients. Also, there is no clearly established theory in the academic community as to on what stage in the progression of glaucoma the retinal ganglion cell death occurs, nor is there any suggestion in this report, nor is the significance of the enhancement discussed. The carnitine included in this report is, contrary to the report, described as a molecule that is upregulated in retinal tissue at an early stage of optic neuropathy in a recent report using a mouse model (Sato, K., Sci Rep., 8, 11930-13 (2018)). These facts call into question the significance in the disease pathogenesis of the metabolites described in both reports. It really suggests strongly the indispensable necessity of validation of the significance with actual human specimens for claims of usefulness in the diagnosis of glaucoma pathogenesis.

In addition, as shown in Test Examples described later, 2 metabolites in NTG, 8 metabolites in POAG, and 44 metabolites in PEG (maybe overlapped due to differences in measurement methods) showed variations specifically associated with each disease state. The metabolites that showed inherent such variations are 2-aminoadipate, mannose, GSH (glutathione), alanine, spermine, asparagine, choline, glutamine, glutamic acid, pyroglutamic acid, acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formyl kynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine 1,5-anhydro-D-sorbitol, fucose, and 2-aminoethanol. Of these, the two species that showed variations specifically associated with NTG are 2-aminoadipic acid and mannose. The eight species that showed variations specifically associated with POAG are GSH (glutathione), alanine, spermine, asparagine, choline, glutamine, glutamic acid, pyroglutamic acid. The 44 species that showed variations specifically associated with PEG (maybe overlapped due to differences in measurement methods) are acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formylkynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine 1,5-anhydro-D-sorbitol, fucose, and 2-aminoethanol. These metabolites can be used as markers to identify distinct glaucoma types (diagnosis marker of the present invention).

Each of the above metabolites may be contained in extracellular vesicles (EVs) in the aqueous humors.

Therefore, by collecting aqueous humors from the subject (patient) and measuring the concentration levels of metabolites that appear in the collected aqueous humors by mass spectrometry (MS), it is possible to diagnose the risk of developing glaucoma development in the pre-disease stage and to diagnose the progression of end-stage glaucoma.

Mass spectrometry methods that may be used in the present invention encompass every technique that allows the measurement of the molecular weight (i.e., mass) or mass variation corresponding to a metabolite. Elemental technologies that constitute such mass spectrometry include sample introduction, ionization, mass separation, ion detection, and recording. Also, the equipment that specifically realizes said mass spectrometry method includes, for example, a mass spectrometer. The mass spectrometer can be a device that comprises a sample introduction section, an ionization section, a mass separation section, a detection section, a recording section, etc., corresponding to each elemental technology of the above mass spectrometry method.

Ionization methods used in the mass spectrometry can include Electron Ionization (EI) method, Chemical Ionization (CI) method, Field Desorption (FD) ionization method, Fast Atom Bombardment (FAB) method, Liquid Secondary Ionization (LSI) method, Matrix-Assisted Laser Desorption/Ionization (MALDI) method, ElectroSpray Ionization (ESI) method, Atmospheric Pressure Chemical Ionization (APCI) method, etc.

When using tandem mass spectrometry, Collision-Induced Dissociation (CID) in which inert gas molecules such as noble gases (e.g., He, Ne, Ar, preferably Ar) or N₂ collide with the charged particles produced by the initial ionization, can also be further applied.

The above mass spectrometry method can be combined with other analytical methods such as chromatography and capillary electrophoresis (CE). Such analytical methods can include, for example, chromatography-mass spectrometry methods such as gas chromatography-mass spectrometry (GC-MS), liquid chromatography-mass spectrometry (LC-MS), supercritical fluid chromatography-mass spectrometry (SFC-MS); and capillary electrophoresis-mass spectrometry (CE-MS). Of these, chromatography-mass spectrometry is preferred, and GC-MS and/or LC-MS is more preferred.

Chromatographic mass spectrometry analysis can be used for Total Ion Chromatogram (TIC) scans and/or Selected Ion Monitoring (SIM) analysis, or for TIC scans and/or Multiple Reaction Monitoring (MRM) analysis. MRM analysis is also called Selected Reaction Monitoring (SRM) analysis, and can be performed in tandem mass spectrometry analysis.

When analyzing a mixture of many metabolites (metabolome), individual metabolites can be identified using scan mode. The identified individual metabolites can then be quantitatively analyzed with high precision in SIM or MRM mode by specifying the mass of the metabolite. Of these, MRM is preferred from the perspective of high-sensitivity analysis.

Specific examples of evaluation in the evaluation method of the present invention can include determining the occurrence of retinal ganglion cell degeneration, determining the preceding susceptibility of retinal ganglion cells to degeneration or cell death, and determining the vulnerability of retinal ganglion cells.

When aiming at providing a practically useful technique for diagnosing the risk of disease development or for diagnosing disease progression, it is thought to be insufficient to merely depend on the comparison between two groups, selected as described above, by the use of logistic regression analysis as described above, or on the selection of metabolites using Venn diagrams, in order to depict the disruption of network organization in the ocular anterior tissue microenvironment, since only a plurality of independent candidate molecules can be selected therefrom. Therefore, as a further technique in the present invention, we applied a biostatistical method that can sort the characteristics among groups as molecular species groups. This method can be applied to the analysis of proteinaceous molecular species present in the ocular anterior tissue microenvironment [cytokines, complement proteins related to innate inflammation, etc.], miRNAs (often contained in exosomes), metabolic products, etc., within each of these molecular groups, as well as to an integrated analysis including these different molecular groups.

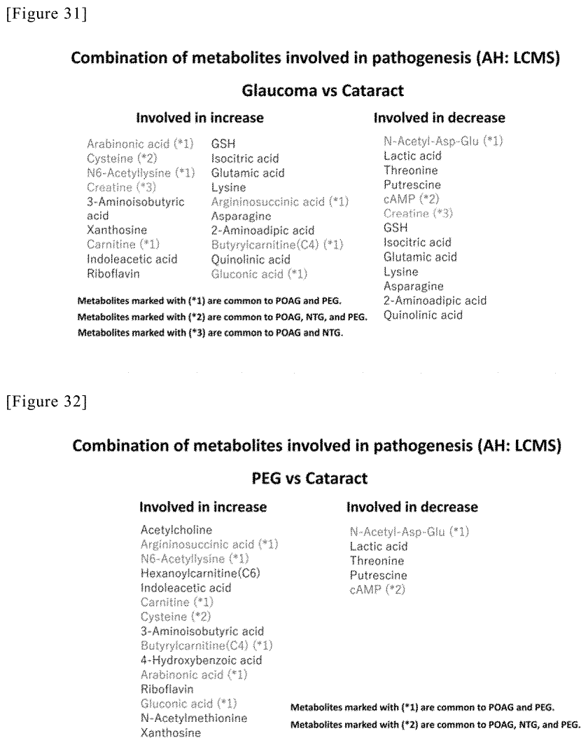

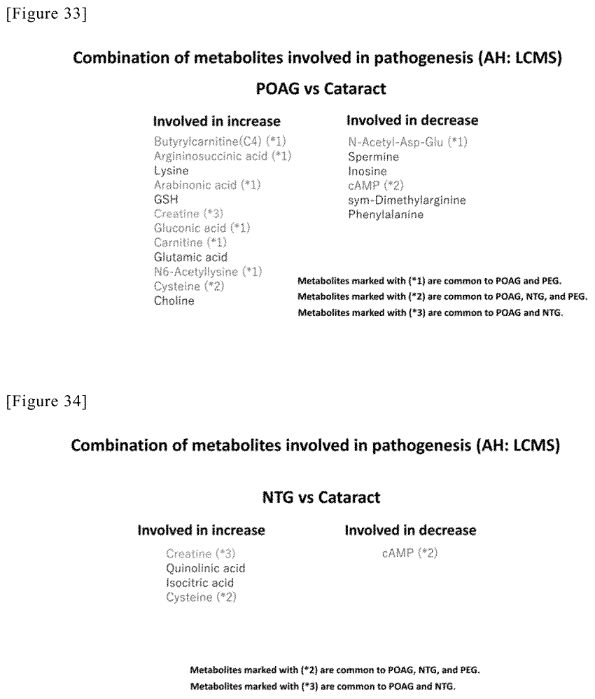

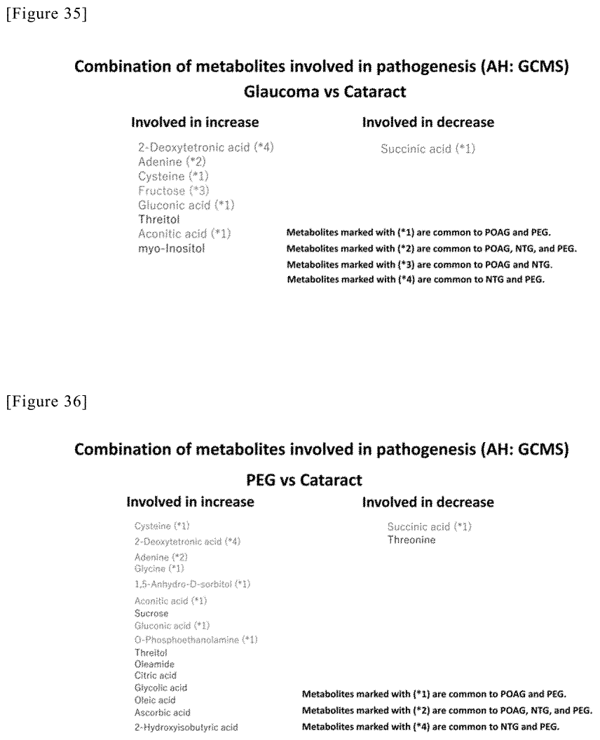

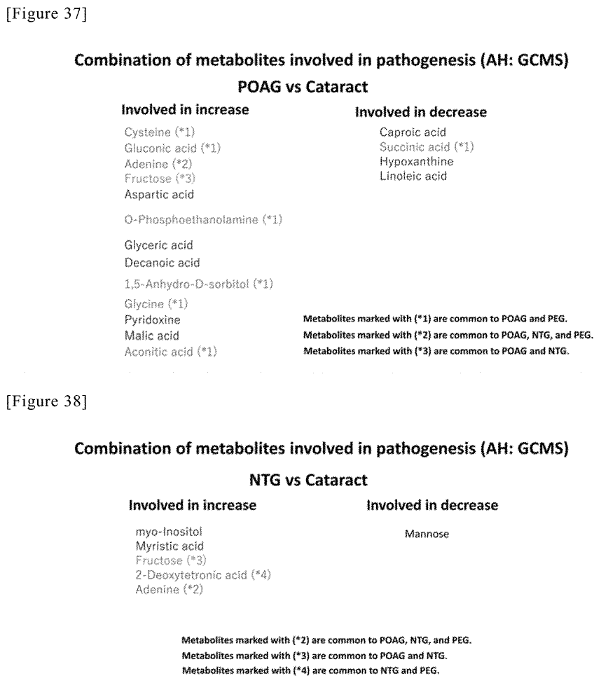

Here, an embodiment is illustrated for metabolites, with the aim of extracting combinations of multiple candidate disease-discriminating markers (metabolites) related to three glaucoma pathological types (POAG, NTG, and PEG). Selection with LASSO (least absolute shrinkage and selection operator), a machine learning method, for all metabolites measured on each of the above-mentioned platforms extracted one pair of models (combinations of plural metabolites and their weights). The number of samples was 28 patients in POAG, 24 patients in NTG, 29 patients in PEG, and 31 patients in cataract. FIGS. 31 to 38 show the results. For markers available for diagnosing the progression of end-stage glaucoma accompanied by cell death of retinal ganglia (evaluation markers of the present invention), which is one of the objectives of the present invention, the results of Lasso analysis in glaucoma vs. cataract may be applicable. Namely, recited as up-regulated factors are the following 19 species: Arabinonic acid, Cysteine, N6-Acetyllysine, Creatine, 3-Aminoisobutyric acid, Xanthosine, Carnitine, Indoleacetic acid, Riboflavin, GSH, Isocitric acid, Glutamic acid, Lysine, Argininosuccinic acid, Asparagine, 2-Aminoadipic acid, Butyrylcarnitine (C4), Quinolinic acid, and Gluconic acid; and down-regulated factors are the following 12 species: N-Acetyl-Asp-Glu, Lactic acid, Threonine, Putrescine, cAMP, Creatine, GSH, Isocitric acid, Glutamic acid, Lysine, Asparagine, 2-Aminoadipic acid, and Quinolinic acid; with a total being 31 species. However, the up-regulated factors common to the three disease types are restricted to Cysteine only, and the down-regulated factors are restricted to cAMP only.

Second, for the diagnosis of early glaucoma risk potential that cannot be detected by current diagnosis techniques, the results of this analysis in comparison to the NTG group can be utilized. Even Lasso analysis with a limited population of 31 cases in the cataract group and 24 cases in the NTG group showed that a “combination” of a total of 8 species, namely, Creatine, Quinolinic acid, Isocitric acid, myo-Inositol, Myristic acid, Fructose, 2-Deoxytetronic acid, and Mannose was chosen, with the discrimination rate of the combination: 76.4%, sensitivity: 66.7%, and specificity: 83.9%.

Thus, while analysis within each of the same molecular species, such as metabolites found in the aqueous humors, can yield meaningful results, it is also possible to apply a biostatistical method, that can sort characteristics between groups as molecular species groups, to different molecular species groups. In other words, the method uses a biostatistical method that allows selection of significant molecular species from among those present in the ocular anterior tissue microenvironment, such as proteinaceous species (e.g., cytokines, complement proteins associated with innate inflammation), miRNAs (often contained in exosomes), and metabolites. Here, embodiments about metabolites, and about miRNAs that vary in the aqueous humors are exemplified.

As described above, it was proven that there exists a vicious cycle for disease progression, in which reduction in miRNA34a-5p and miRNA378a-3p in degenerated human corneal endothelial cells, which is a simulation model of corneal endothelial cell dysfunction in patient tissue, enhances extracellular release of exosomes with encapsulated miRNA23a, miRNA24, miRNA184, etc., which are taken into neighboring cells in a paracrine fashion and SASP (cellular senescence-associated secretory phenotype) is enhanced by the action of the vicious encapsulated miRNAs, and then the SASP causes senescence and degeneration of neighboring cells, and whereby we revealed the possibility that miRNAs released by endothelial cells associated with exosomes as one of the molecular profiles of the ocular anterior tissue microenvironment that underlies the present invention may be involved in the disease pathogenesis, including glaucoma, which is the subject of the present invention, through disruption of the network homeostasis among ocular anterior tissues (see FIG. 16).

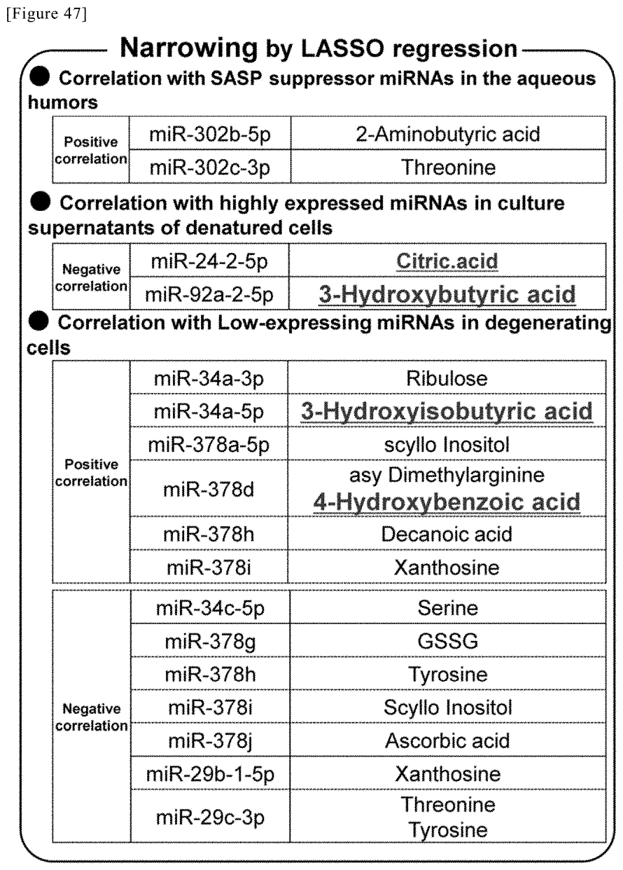

According to this notion, metabolites correlated with miRNAs and their families involved in the pathogenesis of corneal endothelium failures were extracted from the aqueous humors of patients with corneal endothelial failures (bullous keratopathy) (n=29), for which mass spectrometry and miRNA analysis have been performed, and then calculating regression coefficients from Lasso (Least absolute shrinkage and selection operator) with 2565 miRNAs as explanatory variables and 212 metabolites detected in the aqueous humors by mass spectrometry as objective variables (FIG. 46). (1) metabolites correlated with SASP (senescence-associated secretory phenotype) suppressing miRNAs in the aqueous humors: miR-145-5p, miR-302e, miR-1246, and miR-3607-3p, (2) metabolites correlated with miRNAs over-expressed in the culture supernatant of degenerated cells of corneal endothelial cells: miR-23a-3p, miR-24-3p, miR-92b-5p, and miR-184, (3) metabolites correlated with miRNAs repressed in the degenerated cells: miR-34a-5p, miR-378a-3p, miR-29a-3p, miR-29b-3p, and miR-184a-3p, were extracted, and the molecular species shown in FIG. 47 were selected.

Among these, 2-hydroxybutyric acid, 3-hydroxyisobutyric acid, and 4-hydroxybenzoic acid, were selected as metabolites correlated with the clinical phenotypes shown in FIG. 25. These are called aliphatic hydroxy acids, which are biosynthesized from keto acids and are abundant in the TCA cycle, an important energy metabolism pathway in mitochondria, and exquisitely regulate mitochondrial functions.

Thus, the usefulness of the method has been demonstrated, and this usefulness will be further strengthened by further improving the population size. In addition, to further improve this diagnosis technique, it is desirable to include in the analysis proteinaceous molecular species (cytokines, complement system proteins related to innate inflammation, etc.), miRNAs (often contained in exosomes), metabolites, etc., present in the ocular anterior chamber tissue microenvironment, of which the results of analysis have already been described in detail. Such methods are also included in the present invention. This provides a method for diagnosing the risk of ocular disease development, especially ocular diseases caused by disruption of the ocular anterior tissue microenvironment, and for diagnosing the pathological progression of ocular diseases related to disruption of the ocular anterior tissue microenvironment.

The selection of metabolites was performed using LASSO (least absolute shrinkage and selection operator), one of machine learning methods on plasma of patients from whom aqueous humor samples were collected, for all metabolites with the aim of extracting combinations of multiple candidate disease-discriminating markers (metabolites) related to the three glaucoma types presented here (POAG, NTG, and PEG) (Limited test with 20 cases of cataract control, 7 cases of POAG, 9 cases of NTG, 10 cases of PEG). Unlike the results for the aqueous humors described above, the LC-MS test for glaucoma versus cataract showed increases in glutamic acid, anthranilic acid, and taurine, and decreases in indole pyruvic acid, 4-hydroxyproline, and trigonelline. The results of the test were completely different from those of the aqueous humors. Common molecules to three glaucoma disease types could not be selected neither as increased ones nor as decreased ones. This is interpreted as indicating that there is more scientific rationale in glaucoma for diagnosis of aqueous humors as locally interacting body fluid in glaucoma compared to systemically acting body fluids.

In addition to the method described here, the present inventors have obtained target patient population suitable to select patients with a risk of very early glaucoma. These patients can be used and useful to establish the originality and practical usefulness of the present invention.

The present inventors have collected glaucoma specimens from glaucoma specialized outpatient care. The present inventors have collected normal controls by recruiting volunteers and through performing as many multiple detailed examinations and medical examinations as the glaucoma specialized outpatient care. The numbers of glaucoma cases and normal controls have reached 4,400 and 2,400, respectively. In addition to DNA and RNA extractions from these specimens and storage, these specimens have been established as immortalized cell lines with EB virus and have become a semi-permanent bioresource. Using these specimens, we were the first in the world to report the gene for primary open-angle glaucoma (Proc Natl Acad Sci USA. 2009), the first to identify CDKN2BAS-1 as a gene more specifically involved in normal tension glaucoma than in primary open-angle glaucoma (PLoS One. 2012), and identified it as a novel gene specific among Japanese in pseudoexfoliative glaucoma (Sci Rep. 2014, PLoS One. 2012). We have also identified various glaucoma disease-type specific genes for primary open-angle glaucoma (Hum Mol Genet. 2015.), pseudoexfoliative glaucoma (Nat Genet. 2015, Nat. Genet. 2017), and primary angle-closure glaucoma (Nat Genet. 2016). Thus, other susceptibility genes involved in glaucoma development have also been identified, but they are genes relating to of relatively low risk of glaucoma development, and few genes have been mapped to genes responsible for the development of glaucoma or its pathological progressio. This fact clearly shows the limitation of seeking only the risk genes, i.e., it does not meet the practical usefulness. This illustrates the practical utility of the present invention to depict disease-type-specific molecules in regard to related diseases related to the disruption of the ocular anterior tissue microenvironment. In addition, the present invention discloses a new technology which can diagnose disruption of the ocular anterior tissue microenvironment at the molecular level, with a comprehensive overview of proteinaceous molecules including cytokines, related to the composition of functional networks among ocular anterior tissues, nucleic acid molecules such as miRNAs that regulate acquired gene expression of genes (epigenetic expression), and metabolites whose production is regulated by the interaction of these molecular groups. Both together provide a novel and practical technology for diagnosing the risk of disease development or for diagnosing disease progression.

Various environmental factors are the candidates for risk factors for the development of glaucoma. Aging decreases the number of retinal ganglion cells, intraocular pressure increases with cold, and oxidative stress causes trabecular meshwork dysfunction and changes in aqueous humor composition, which aggravate molecular profiles in aqueous humors and cause increased IOP, leading to damages to the axons and retinal ganglion cells as well. The prevalence of glaucoma increases with age, and current technology cannot guarantee lifelong symptom-free normality. A “glaucoma resistant group, namely normal control that remains symptom-free lifelong time” is needed, and the inventors have the advantage of having this specialized patient group. Only by comparing the normal control with the age-corrected glaucoma group, we can estimate lifetime resistance to the development of the disease. The present invention also includes the technologies of using cases that have remained normal for at least 10 years as controls. By using as controls those cases that have remained normal for 10 years, which has first become possible with the accumulation of these technologies by the present inventors, the application of the technologies of the present invention will firstly lead to a revolutionary evaluation technology that reflect the disruption of ocular anterior tissue microenvironment in the pre-disease stage of the glaucoma.

Thus, the provision of a molecular diagnosis technique for disease type stratification that combines the information obtained in conventional medical care including optical methods and the genomic information, in addition to the comprehensive analysis of cytokines, miRNAs, and metabolites in the aqueous humors, which is the diagnosis technology of the present invention, will contribute to the realization of appropriate medical care for patients with eye diseases caused by a disruption of the ocular anterior tissue microenvironment, especially the diseases which are commonly called glaucoma, and to the establishment of an international standard diagnosis method for the risk of development or for the progression of the disease.

In addition, the diagnosis method of the present invention can be used in combination with other analysis results, findings, information, etc., to perform more accurate evaluation. Such analytical results include, for example, the following:
Results of molecular profile analysis of metabolites in patients with corneal endothelial failures by comprehensive metabolome analysis (metabolomics);
Results of analysis of proteinaceous molecular profiles by comprehensive proteome analysis (proteomics);
Findings from gene expression analysis using proteomics and/or clinical specimens;
Information pertaining to factors in tear fluid, blood, and/or other body fluids; and the comprehensive medical information analysis with the above results and others is also included in the present invention.

### 2. Diagnosis System According to the Present Invention

The diagnosis system according to the present invention (hereinafter referred to as the “diagnosis system of the present invention”) is a diagnosis system for the risk of the development of an ocular disease or for the progression of an end-stage ocular disease, which is used in the diagnosis method of the present invention, comprising a means for collecting an aqueous humor sample and a means for analyzing the sample by mass spectrometry (MS).

The specimen collection means used in the diagnosis system of the present invention can include, for example, a pipette.

The analytical means used in the diagnosis system of the present invention can include, for example, a mass spectrometer.

The mass spectrometer can be a device that includes a sample introduction section, ionization section, mass separation section, detection section, and recording section.

As the mass separation section, a double-focusing mass spectrometer (magnetic field mass spectrometer), Quadrupole Mass Spectrometer (Q MS), ion trap (IT) mass spectrometer, Time-Of-Flight (TOF) mass spectrometer, Fourier Transform Ion Cyclotron Resonance (FT-ICR) mass spectrometer, etc. can be used.

The mass separation section may be a single mass spectrometer or a tandem mass spectrometer. The mode of the tandem mass spectrometer is not limited, and, for example, triple quadrupole mass spectrometer, quadrupole-time-of-flight (Q-TOF) mass spectrometer, ion trap-time-of-flight (IT-TOF) mass spectrometer, and tandem TOF mass spectrometers can be used. Among these, the triple quadrupole mass spectrometer is preferred.

The above analytical means can be combined with other analytical means such as chromatographs, capillary electrophoresis (CE) devices, etc. Such analytical means can include, for example, chromatograph mass spectrometers such as Gas Chromatograph/Mass Spectrometer (GC-MS), Liquid Chromatograph/Mass Spectrometer (LC-MS), Supercritical Fluid Chromatograph/Mass Spectrometer (SFC-MS); and Capillary Electrophoresis/Mass Spectrometer: CE-MS). Among these, chromatograph-mass spectrometers are preferred, and GC-MS and/or LC-MS is more preferred.

### EXAMPLE

Hereinafter, the present invention will be described with reference to Test Examples or Examples, but the present invention is not limited by these Examples in any way.

### Test Example: Comprehensive Metabolite Analysis

Using aqueous humors collected from 31 cataract patients, 81 glaucoma patients, and 62 patients with corneal endothelial damage, a comprehensive metabolite analysis (metabolomic analysis) was performed by means of GC-MS and LC-MS. A volume of 100 μL of aqueous humors was collected from each patient using a dedicated pipette.

The glaucoma patient group (81 patients) included 24 patients with normal tension glaucoma (NTG), 28 patients with primary open-angle glaucoma (POAG), and 29 patients with pseudoexfoliative glaucoma (PEG). Glaucoma specimens were collected at the time of surgery from glaucoma cases with apparent visual field defects.

The corneal endothelial damage group (62 patients) includes 41 patients who underwent corneal endothelial transplantation (corneal transplant group) and 21 patients who received cell injection by corneal endothelial regenerative medicine (cultured human corneal endothelial cell injection therapy) (cell injection group).

### (1) Measurement by Gas Chromatograph-Mass Spectrometer

### <Sample Preparation>

First, 720 μL of an internal standard solution (2-isopropyl malate 0.1 mg/mL) was added to 30 mL of methanol and mixed well (methanol with internal standard). Take 30 μL of the aqueous humor sample in a tube, add 154 μL of methanol with internal standard, and immediately mixed vigorously with a tabletop mixer. After mixing for 10 minutes under a condition of 37° C. and 1200 rpm in a thermostatic shaker, centrifugation was performed, and 120 μL of the supernatant only was collected in a new micro tube to remove proteins.

Next, 72 μL of 1% acetic acid and 96 μL of chloroform were added to the supernatant obtained, and after the mixture was mixed with a tabletop mixer for 15 seconds, the mixture was centrifuged again. The hydrophilic metabolites were then extracted by collecting 60 μL of the upper layer in a new micro tube.

The resulting upper layer was then evaporated using a centrifugal evaporator to vaporize the solvent, and then dried by lyophilization to remove water.

Finally, 40 μL of methoxyamine pyridine solution (20 mg/mL) was added to the lyophilized samples for oxime derivatization (shaking at 37° C. for 30 min.), followed by adding 20 μL of MSTFA for trimethylsilyl derivatization (shaken at 37° C. for 30 min.).

### <Measuring Device>

The above samples were analyzed using a single quadrupole gas chromatograph mass spectrometer (GCMS-QP 2010Ultra, manufactured by Shimadzu Corporation) and data was collected. The analysis was performed under the following conditions.

### <Measurement Conditions>

GC section: The sample injection volume was 1 μL, and DB-5 (30 m×0.25 mm I.D., df=1 μm, capillary column manufactured by Agilent) was used as a column. The column oven temperature was maintained at 80° C. for 2.5 min after the start of measurement, and then increased to 280° C. at a rate of 12.5° C./min. The attained temperature of 280° C. was maintained for 4.5 minutes. The vaporization chamber temperature was set at 280° C., and the linear velocity of the carrier gas (He) was set at 39 cm/sec.
MS section: Interface temperature and ion source temperature were set to 200° C. and 250° C., respectively. Quantitative analysis was performed in scan mode.

### (2) Measurement by Liquid Chromatography-Mass Spectrometer

### <Sample Preparation>

The aqueous humors of the specimens were aliquoted into microtubes, to which 2-isopropyl malate aqueous solution was added. The further process of protein removal is the same as for GC-MS analysis.

Next, the process for extraction of hydrophilic metabolites is also the same as in GC-MS analysis.

Then, in the same manner again, the solvent was vaporized using a centrifugal evaporator and then dried by the lyophilization method to remove water.

Finally, 25 μL of 0.1% formic acid aqueous solution was added to the lyophilized specimen, mixed well, and dispensed into a vial as a sample.

### <Measuring Device>

The above samples were analyzed by LC-MS using a high-performance liquid chromatograph (Nexera, manufactured by Shimadzu Corporation) as the LC section and a triple quadrupole mass spectrometer (LCMS-8040, manufactured by Shimadzu Corporation) as the MS section to collect data. The analysis was performed under the following conditions.

### <Measurement Conditions>

LC section: The sample injection volume was 4 μL, and Shim-pack GIST C18-AQ (250 mm I.D.×2.1 mL, 3 μm, reversed phase column manufactured by Shimadzu GLC) was used as a column. The column oven temperature was set at 40° C. and the flow rate of the mobile phase was set at 0.2 mL/min. Two mobile phases of A (mixed solvent of formic acid:water=1:1000 by volume) and B (mixed solvent of formic acid:acetonitrile=1:1000 by volume) were used as mobile phases, and the mixing ratio of both mobile phases was changed sequentially over time to perform the gradient analysis. The time program of the gradient analysis is as follows. 0% B (0 to 3.0 min.)→60% B (15.0 min.)→95% B (15.01 min.)→95% B (15.01 to 20.0 min.)→0% B (20.1 to 25.0 min.)
MS section: ESI (Positive/Negative) was used as the ionization method. Compound identification was performed in scan mode, and quantitative analysis was performed in MRM mode. The following setting values were used for each analysis.

Nebulizer gas flow rate: 3 L/min

Drying gas flow rate: 15 L/min

DL temperature: 250° C.

Heat block temperature: 400° C.

### (3) Analysis of Measurement Results

(3-1) Analysis of Glaucoma Group Data with Cataract Group as Control

96 metabolites were identified in the GC-MS analysis and 114 metabolites were identified in the LC-MS analysis, and their peak area values were calculated. The peak area value of each metabolite was divided by the peak area value of an internal standard (2-isopropyl malic acid) of which a fixed amount was added to each of the samples, and the resultant value was adopted as the semi-quantitative value reflecting the concentration in the aqueous humors and compared in each group.

The cataract group was used as a control and compared with each of the three disease-type distinct glaucoma groups (NTG, POAG and PEG) by Wilcoxon rank sum test. The results showed that there were statistically significant differences (p-value <0.05) in 22 metabolites in comparison with the NTG group, in 54 metabolites in comparison with the POAG group, and in 83 metabolites in comparison with the PEG group. Of these, 15 metabolites were variable in common among the three disease types.

The list of the 15 metabolites, the ratio of means in the comparison with the control group (Fold Change: FC) and p-values are shown in Table 1. In addition, as the metabolites that show specific variation in each disease-type group, there identified 2 metabolites in the NTG group, 8 metabolites in the POAG group, and 44 metabolites in the PEG group. The list of these metabolites, and FC values and p-values in comparison to the control group are shown in Table 2.

These metabolites can be used as markers for discriminating glaucoma disease types (diagnosis marker of the present invention).

TABLE 1

| | | FC | p-value | | FC | p-value | | FC | p-value |
|---|---|---|---|---|---|---|---|---|---|
| Arabinonic acid(LCMS) | PEG | 1.62 | 6.89E−06 | NTG | 1.51 | 8.54E−05 | POAG | 1.47 | 2.77E−04 |
| Arabinonic acid(GCMS) | PEG | 1.51 | 2.30E−04 | NTG | 1.53 | 9.16E−05 | POAG | 1.44 | 1.26E−03 |
| myo-Inositol | PEG | 1.16 | 2.36E−02 | NTG | 1.27 | 7.10E−04 | POAG | 1.16 | 4.43E−02 |
| cAMP | PEG | 0.81 | 4.75E−02 | NTG | 0.69 | 2.59E−03 | POAG | 0.78 | 1.15E−02 |
| Fructose | PEG | 1.16 | 2.10E−02 | NTG | 1.19 | 1.01E−02 | POAG | 1.37 | 5.23E−04 |
| asy-Dimethylarginine | PEG | 1.30 | 2.49E−05 | NTG | 1.17 | 1.49E−02 | POAG | 1.20 | 1.63E−03 |
| Citric acid | PEG | 2.18 | 1.46E−05 | NTG | 1.45 | 1.56E−02 | POAG | 2.28 | 4.87E−03 |
| Quinolinic acid | PEG | 2.06 | 2.33E−05 | NTG | 1.23 | 1.63E−02 | POAG | 1.56 | 8.63E−04 |
| Cysteine | PEG | 2.63 | 4.55E−07 | NTG | 1.39 | 2.35E−02 | POAG | 2.07 | 8.18E−05 |
| Spermidine | PEG | 0.61 | 1.26E−03 | NTG | 0.71 | 2.56E−02 | POAG | 0.66 | 5.35E−03 |
| Carnitine | PEG | 1.49 | 1.51E−07 | NTG | 1.13 | 2.68E−02 | POAG | 1.35 | 9.00E−06 |
| Isobutyrylcarnitine(C4) | PEG | 2.09 | 1.13E−06 | NTG | 1.21 | 3.05E−02 | POAG | 2.04 | 3.30E−05 |
| 3-Methylhistidine | PEG | 1.60 | 6.68E−04 | NTG | 1.17 | 3.92E−02 | POAG | 1.27 | 1.91E−03 |
| Propionylcarnitine | PEG | 1.82 | 2.37E−06 | NTG | 1.21 | 4.09E−02 | POAG | 1.58 | 2.62E−04 |
| Isocitric acid | PEG | 1.87 | 2.90E−04 | NTG | 1.33 | 4.43E−02 | POAG | 1.56 | 2.72E−03 |

TABLE 2

| | | FC | p-value | | FC | p-value | | FC | p-value |
|---|---|---|---|---|---|---|---|---|---|
| NTG inherency | | | | | | | | | |
| 2-Aminoadipic acid | PEG | 1.18 | 7.59.E−02 | NTG | 1.20 | 2.68.E−02 | POAG | 1.21 | 9.65.E−02 |
| Mannose | PEG | 0.89 | 2.20.E−01 | NTG | 0.81 | 3.32.E−02 | POAG | 1.21 | 1.65.E−01 |
| POAG inherency | | | | | | | | | |
| GSH | PEG | 2.71 | 3.07.E−01 | NTG | 2.61 | 3.73.E−01 | POAG | 6.39 | 3.12.E−04 |
| Alanine | PEG | 1.09 | 5.64.E−02 | NTG | 1.09 | 1.72.E−01 | POAG | 1.22 | 5.86.E−04 |
| Spermine | PEG | 0.88 | 1.83.E−01 | NTG | 0.83 | 1.01.E−01 | POAG | 0.70 | 1.40.E−03 |
| Asparagine | PEG | 1.12 | 1.24.E−01 | NTG | 1.05 | 4.01.E−01 | POAG | 1.12 | 1.42.E−02 |
| Choline | PEG | 1.04 | 4.69.E−01 | NTG | 1.00 | 6.29.E−01 | POAG | 1.27 | 1.98.E−02 |
| Glutamine | PEG | 1.01 | 5.74.E−01 | NTG | 1.05 | 3.29.E−01 | POAG | 1.10 | 2.62.E−02 |
| Glutamic acid | PEG | 1.18 | 6.66.E−02 | NTG | 1.01 | 6.05.E−01 | POAG | 1.16 | 3.42.E−02 |
| Pyroglutamic acid(GCMS) | PEG | 1.05 | 2.80.E−01 | NTG | 1.12 | 1.61.E−01 | POAG | 1.12 | 4.93.E−02 |
| PEG inherency | | | | | | | | | |
| Acetylcholine | PEG | 2.01 | 1.23.E−08 | NTG | 1.12 | 4.71.E−01 | POAG | 1.33 | 5.68.E−02 |
| Xanthosine | PEG | 2.08 | 2.66.E−05 | NTG | 1.16 | 5.70.E−01 | POAG | 1.37 | 1.03.E−01 |
| N-Acetylarginine | PEG | 1.58 | 5.05.E−05 | NTG | 0.92 | 4.81.E−01 | POAG | 1.21 | 2.85.E−01 |
| Glycine | PEG | 2.29 | 7.36.E−05 | NTG | 1.39 | 6.81.E−02 | POAG | 1.44 | 2.39.E−01 |
| 3-Aminoisobutyric acid | PEG | 2.30 | 1.20.E−04 | NTG | 1.09 | 2.97.E−01 | POAG | 1.32 | 3.91.E−01 |
| Cystine(GCMS) | PEG | 2.32 | 5.08.E−04 | NTG | 1.27 | 2.85.E−01 | POAG | 1.50 | 1.09.E−01 |
| Kynurenine | PEG | 1.50 | 6.33.E−04 | NTG | 0.95 | 8.32.E−01 | POAG | 1.22 | 5.48.E−02 |
| Kynurenic acid | PEG | 2.59 | 7.19.E−04 | NTG | 1.10 | 7.16.E−01 | POAG | 1.68 | 7.63.E−02 |
| 4-Hydroxyhippuric acid | PEG | 2.22 | 7.81.E−04 | NTG | 1.07 | 1.56.E−01 | POAG | 1.32 | 3.41.E−01 |
| 4-Hydroxyproline | PEG | 1.50 | 8.28.E−04 | NTG | 0.89 | 6.53.E−01 | POAG | 1.15 | 1.51.E−01 |
| 4-Hydroxyproline(GCMS) | PEG | 1.42 | 2.31.E−03 | NTG | 0.91 | 6.41.E−01 | POAG | 1.18 | 8.22.E−02 |
| Pyridoxic acid | PEG | 4.75 | 2.80.E−03 | NTG | 0.68 | 3.94.E−01 | POAG | 0.41 | 8.67.E−02 |
| Isocitric acid(GCMS) | PEG | 1.75 | 3.40.E−03 | NTG | 1.06 | 6.90.E−01 | POAG | 1.25 | 2.13.E−01 |
| N-Acetylglucosamine | PEG | 1.32 | 3.40.E−03 | NTG | 1.14 | 1.29.E−01 | POAG | 1.12 | 9.05.E−02 |
| GSSG | PEG | 270.97 | 5.39.E−03 | NTG | 1.06 | 9.11.E−01 | POAG | 70.12 | 5.94.E−01 |
| N′-Formylkynurenine | PEG | 1.47 | 6.49.E−03 | NTG | 0.92 | 7.66.E−01 | POAG | 1.07 | 4.71.E−01 |
| Creatinine | PEG | 1.31 | 7.75.E−03 | NTG | 1.03 | 4.81.E−01 | POAG | 1.10 | 9.96.E−02 |
| N-Acetylmethionine | PEG | 1.16 | 9.23.E−03 | NTG | 0.93 | 2.81.E−01 | POAG | 1.06 | 2.28.E−01 |
| Ornithine | PEG | 1.18 | 9.23.E−03 | NTG | 0.87 | 3.82.E−01 | POAG | 1.07 | 1.84.E−01 |
| Citrulline(GCMS) | PEG | 1.29 | 9.23.E−03 | NTG | 0.98 | 9.93.E−01 | POAG | 1.06 | 2.98.E−01 |
| Oleamide | PEG | 1.53 | 9.63.E−03 | NTG | 1.09 | 7.79.E−01 | POAG | 1.25 | 3.99.E−01 |
| Arabitol | PEG | 0.98 | 1.09.E−02 | NTG | 0.91 | 1.09.E−01 | POAG | 0.90 | 3.28.E−01 |
| Adenosylhomocysteine | PEG | 1.49 | 1.14.E−02 | NTG | 1.09 | 4.30.E−01 | POAG | 1.03 | 2.05.E−01 |
| Hippuric acid | PEG | 2.64 | 1.22.E−02 | NTG | 1.47 | 4.06.E−01 | POAG | 1.32 | 9.88.E−01 |
| trans-urocanic acid | PEG | 1.72 | 1.25.E−02 | NTG | 1.37 | 5.02.E−01 | POAG | 1.37 | 3.52.E−01 |
| Sucrose | PEG | 1.68 | 1.35.E−02 | NTG | 0.89 | 2.45.E−01 | POAG | 1.01 | 4.43.E−01 |
| Putrescine | PEG | 0.82 | 1.47.E−02 | NTG | 0.85 | 5.62.E−02 | POAG | 1.02 | 4.80.E−01 |
| Urea | PEG | 1.12 | 1.47.E−02 | NTG | 1.05 | 1.25.E−01 | POAG | 1.06 | 1.79.E−01 |
| Succinic acid(GCMS) | PEG | 0.77 | 1.53.E−02 | NTG | 1.11 | 8.59.E−01 | POAG | 0.94 | 6.54.E−01 |
| Riboflavin | PEG | 1.70 | 1.59.E−02 | NTG | 1.18 | 7.03.E−01 | POAG | 1.20 | 2.65.E−01 |
| 2-Hydroxyglutaric acid | PEG | 1.47 | 1.87.E−02 | NTG | 1.17 | 2.00.E−01 | POAG | 1.23 | 2.52.E−01 |
| Malic acid | PEG | 1.41 | 1.94.E−02 | NTG | 1.06 | 8.59.E−01 | POAG | 1.17 | 2.33.E−01 |
| Hypotaurine | PEG | 1.46 | 2.10.E−02 | NTG | 0.89 | 7.34.E−01 | POAG | 1.12 | 3.51.E−01 |
| Urea(GCMS) | PEG | 1.27 | 2.55.E−02 | NTG | 1.04 | 4.40.E−01 | POAG | 1.07 | 2.85.E−01 |
| Pipecolinic acid | PEG | 1.70 | 2.55.E−02 | NTG | 0.77 | 8.06.E−01 | POAG | 1.04 | 1.43.E−01 |
| Guanidinoacetic acid | PEG | 1.34 | 2.65.E−02 | NTG | 0.99 | 8.45.E−01 | POAG | 0.97 | 5.90.E−01 |
| Acetylcarnosine | PEG | 1.93 | 2.97.E−02 | NTG | 0.92 | 5.36.E−01 | POAG | 1.20 | 2.46.E−01 |
| 2-Oxoglutaric acid(GCMS) | PEG | 0.80 | 2.97.E−02 | NTG | 1.11 | 8.06.E−01 | POAG | 1.13 | 5.90.E−01 |
| 3-Hydroxyisovaleric acid(GCMS) | PEG | 1.05 | 3.32.E−02 | NTG | 1.11 | 4.40.E−01 | POAG | 1.06 | 2.65.E−01 |
| Maltose | PEG | 1.48 | 3.75.E−02 | NTG | 0.88 | 4.62.E−01 | POAG | 1.06 | 4.56.E−01 |
| Uridine | PEG | 0.89 | 3.84.E−02 | NTG | 1.01 | 9.66.E−01 | POAG | 0.97 | 9.82.E−01 |
| 1,5-Anhydro-D-sorbitol | PEG | 1.41 | 4.12.E−02 | NTG | 1.12 | 4.20.E−01 | POAG | 1.07 | 5.29.E−01 |
| Fucose | PEG | 1.22 | 4.12.E−02 | NTG | 0.99 | 9.12.E−01 | POAG | 1.10 | 3.91.E−01 |
| 2-Aminoethanol | PEG | 1.25 | 4.58.E−02 | NTG | 1.16 | 2.59.E−01 | POAG | 1.04 | 5.80.E−01 |

(3-2) Performance Evaluation of the Metabolites Selected with Reference to Corneal Endothelial Damage Group Data as Markers for Evaluating the Risk of Developing Glaucoma or for Evaluating the Disease Progression

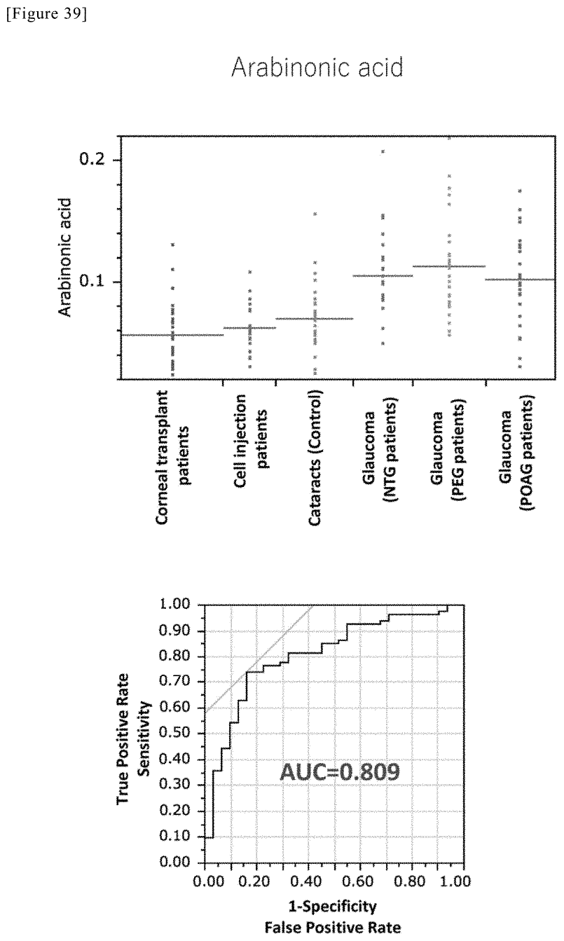

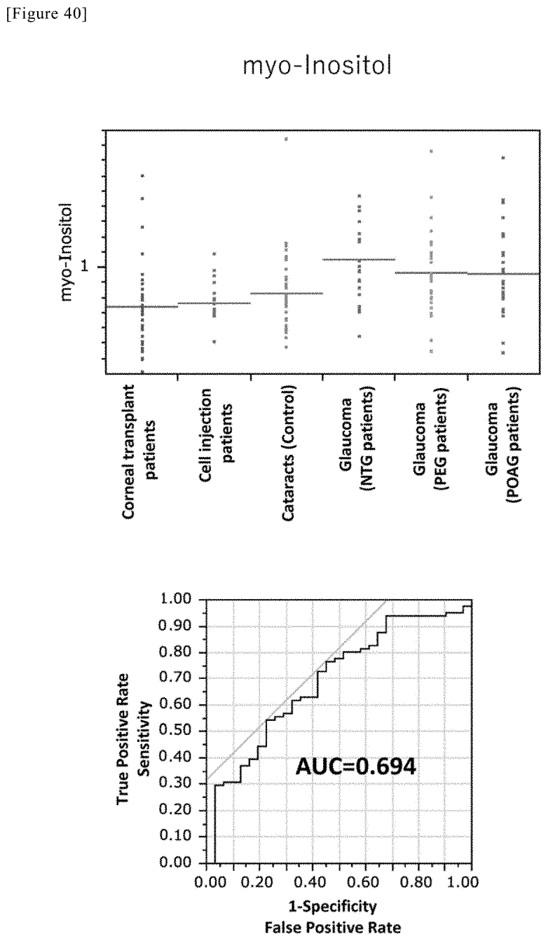

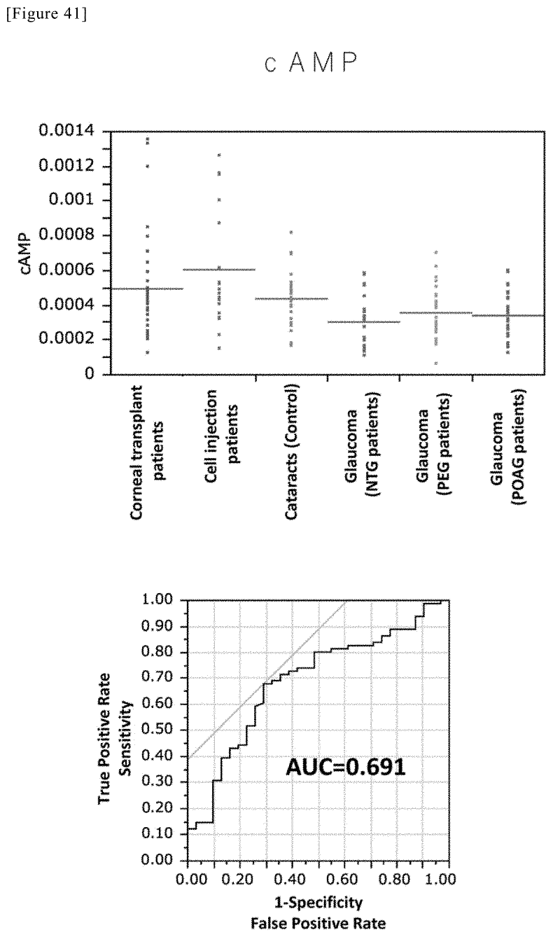

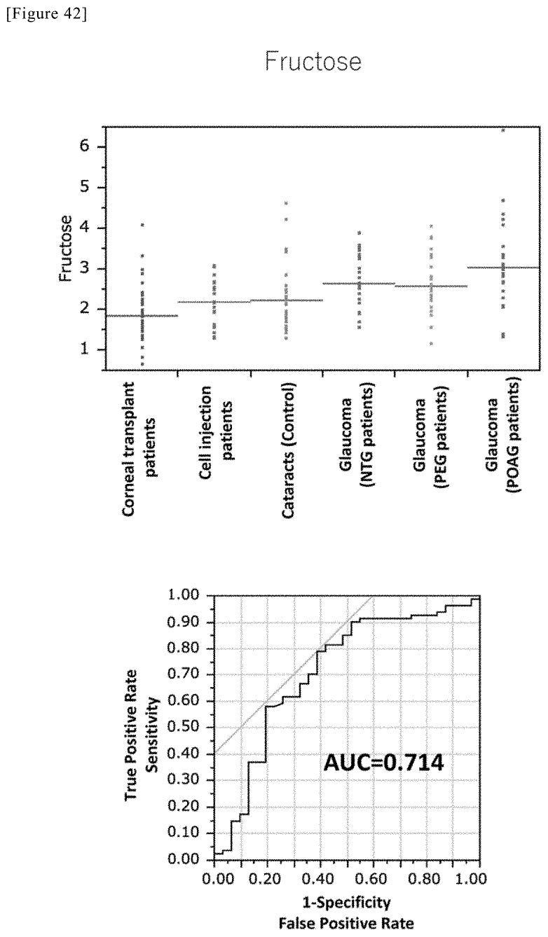

Among the 15 metabolites that were found to vary in common among the three glaucoma disease types above, arabinonic acid, myo-inositol, cAMP, and fructose were found out as the metabolites that may also further differ from the corneal endothelial damage group (corneal transplant and cell injection specimens). The marker performance of these four metabolites was evaluated with cataract being considered as negative and all glaucoma regardless of the cause as positive. The results are shown in FIGS. 39 to 42.

FIGS. 39 to 42 show the results of the amount of each metabolite in the aqueous humors for the corneal endothelial damage group (corneal transplant and cell injection groups), the cataract group, and the glaucoma group, which were quantified by GC-MS or LC-MS. The figures below are the ROC (Receiver Operating Characteristic) curves when each metabolite is used as a marker for glaucoma evaluation. The AUC (Area Under Curve) values are shown in each figure.

These results show that a relatively high true positive rate is achieved for any of the metabolites as a marker, even when the false positive rate value is small. The value of each AUC is also high, especially for arabinoic acid, which has the best value, 0.809.

### (3-3) Performance Evaluation of Markers by Multiple Logistic Regression Analysis

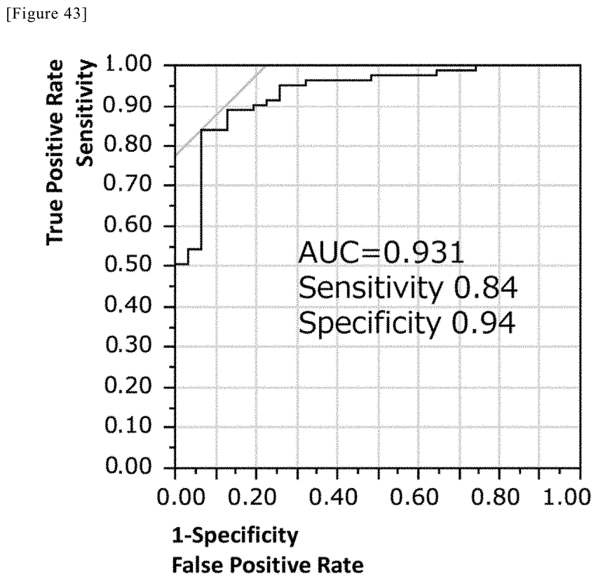

Among the 15 metabolites found to vary in common among the above three glaucoma disease-types, in order to find combinations that discriminate glaucoma groups with further high accuracy, we examined predictive models based on multiple logistic regression analysis. The optimal selection of metabolites was performed by the variable increase/decrease method. As a result, arabinonic acid, cAMP, cysteine, spermidine, and isobutyrylcarnitine (C4) were selected. The regression coefficients of the predictive model created by these five metabolites are shown in Table 3. The marker performance was evaluated by ROC analysis with cataract being considered as negative and all glaucoma regardless of cause as positive, giving a good result with an AUC of 0.931, sensitivity of 0.84, and specificity of 0.94 (FIG. 43).

TABLE 3

| | Estimated regression coefficients | p-value |
|---|---|---|
| Intercept | −3.2 | 0.049 |
| Arabinonic acid | 29.5 | 0.026 |
| Isobutyrylcarnitine (C4) | 42.7 | 0.009 |
| Spermidine | −120.4 | 0.033 |
| Cysteine | 1828.0 | 0.008 |
| cAMP | −4094.9 | 0.084 |

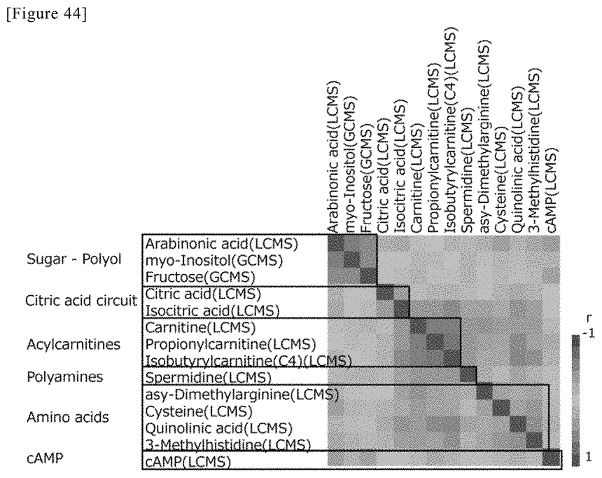

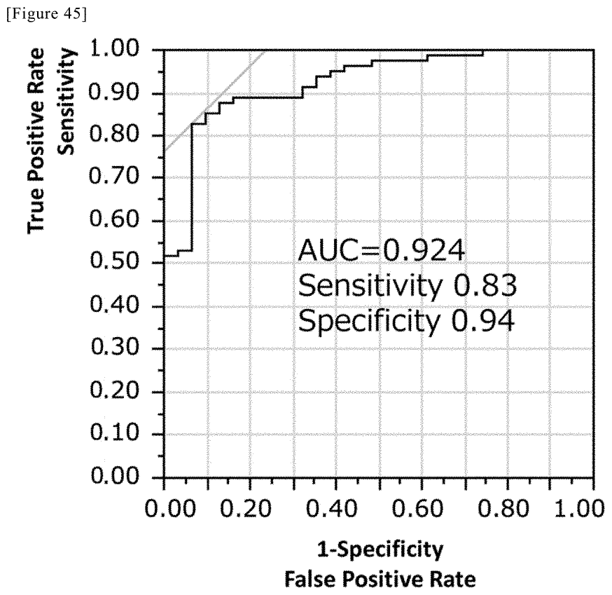

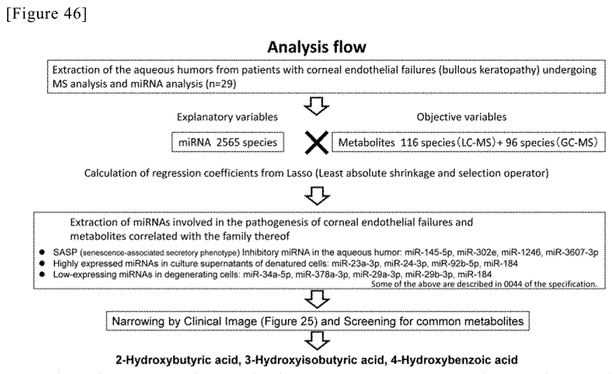

In addition, Pearson product-rate correlation coefficients among these 15 metabolites are shown by color map (FIG. 44). These metabolites were classified into sugar/polyol (arabinonic acid, myo-inositol, fructose), citric acid cycle (citric acid, isocitric acid), amino acids (asy-dimethylarginine, quinolinic acid, cysteine, 3-methylhistidine), polyamines (spermidine), and acylcarnitines (carnitine, isobutyrylcarnitine (C4), propionylcarnitine), and cAMP using the height of correlation as an indicator. For the metabolites selected in the above predictive model, one from each group is selected, and the marker performance is maintained even when the metabolite is replaced by a metabolite within a group. For example, when arabinonic acid is replaced with myo-inositol, the regression coefficients of the predictive model generated are as shown in Table 4. The performance of the predictive model created had an AUC of 0.924, sensitivity of 0.83, and specificity of 0.94, which was a good result (FIG. 45).

TABLE 4

| | Estimated regression coefficients | p-value |
|---|---|---|
| Intercept | −3.3 | 0.074 |
| myo-Inositol | 2.2 | 0.108 |
| Isobutyrylcarnitine (C4) | 52.4 | 0.002 |
| Spermidine | −122.6 | 0.026 |
| Cysteine | 2173.7 | 0.001 |
| cAMP | −4968.1 | 0.026 |

(3-4) Metabolome Analysis with Reference to Corneal Endothelial Damage Group Data

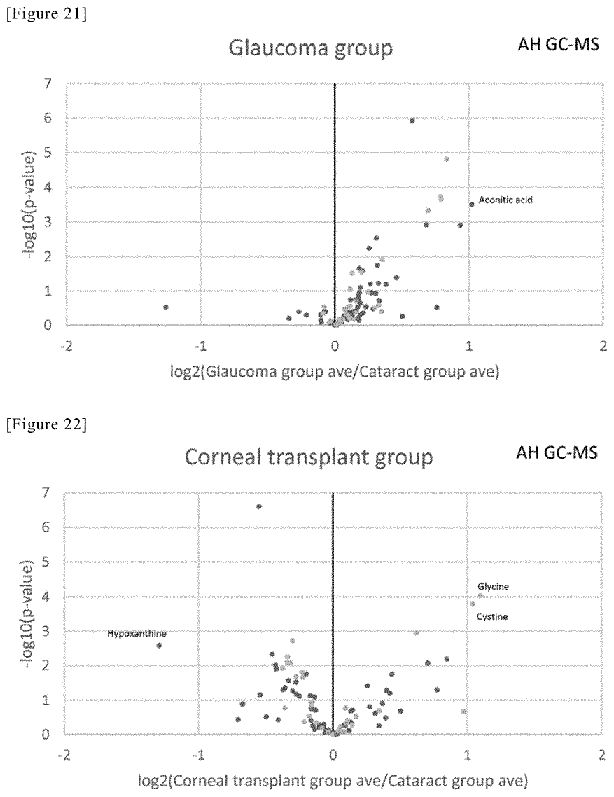

In addition, the statistical significance (p-value) using the Wilcoxon rank-sum test with the cataract group as the control, and the ratio of the mean of each group to the mean of the cataract group (Fold Change: FC) were analyzed comprehensively, for the metabolite amounts in the aqueous humors in the PEG group, corneal transplant group, and cell injection group. The results are shown in FIGS. 18 to 23 (note that the PEG group is labeled as glaucoma in the figures). Of these, FIGS. 18 to 20 shows Volcano plots based on LC-MS measurements, and FIGS. 21 to 23 shows Volcano plots based on GC-MS measurements. The light-colored dots in each figure indicate amino acid metabolites, and the dark-colored dots indicate other metabolites. Note that as for carnitines and metabolites within the range of
−1.0<log₂ (FC)<1.0, and 1.3<log₁₀ (p-value),
   the names are described in each figure.

Among these, for example, looking at the PEG group, it is found that, in the metabolites measured by LC-MS, carnitine shows a high level of significance against the cataract group. The metabolite that shows both a concentration difference and a significant difference with respect to the cataract group is found to be reduced glutathione (GSH). It is also found that, in the metabolites measured by GC-MS, aconitic acid has both the concentration difference and the significant difference.

(3-5) Extraction of Metabolites Correlated with miRNAs and their Families Involved in the Pathogenesis of Corneal Endothelial Failures by Lasso (Least Absolute Shrinkage and Selection Operator) Regression Method.

Lasso (Least absolute shrinkage and selection operator) regression was used to extract metabolites correlated with miRNAs and their families involved in the pathogenesis of corneal endothelial failures. This method is one of the feature selection methods to select the explanatory variables X necessary to explain the objective variables Y using L1 regularization, and the model is selected by minimizing the following equation.

$${\sum }_{i = 1}^{N} {( {y}_{i} - {\hat{y}}_{i} )}^{2} + λ {\sum }_{k = 1}^{K} \vert {β}_{k} \vert \qquad [ Formula 1 ]$$

i: Sample (1, . . . , N),
k: Number of explanatory variables (1, . . . , K),
y: Objective variables, ŷ: Predicted value,)
β: Partial regression coefficient,
λ: Parameters that determine the effect of regularization.

Here, the aqueous humors of patients with corneal endothelial failures (bullous keratopathy) (n=29), which have been subjected to mass spectrometry and miRNA analysis, were targeted. For each of the 212 metabolites as the objective variable, the analysis was to mathematically obtain one pair of metabolites that contribute to the objective variable from the 2565 miRNAs as explanatory variables.

In the analytical data, if there were missing values of 80% or more in the metabolites of the objective variable, they were excluded from the analysis, and for the complementation of other missing values, 0 was substituted for metabolites and 0.1 for miRNAs. Then, the objective variable and the explanatory variable were binarized (0 or 1) at the respective medians, and 10-fold cross-validation was performed to determine the optimum value for parameter X.

Note that model selection by Lasso regression makes the weights (regression coefficients) of the coefficients of explanatory variables that do not contribute to the objective variable zero, and thus enables the extraction of contributing explanatory variables by reducing the number of explanatory variables simultaneously with the model selection.

Regression coefficients were calculated for one pair of metabolites selected for the model by this analysis method, and metabolites correlated with miRNAs and their families involved in the pathogenesis of corneal endothelial failures were extracted (FIG. 46). Namely, (1) metabolites that correlated with SASP (senescence-associated secretory phenotype) suppressor miRNAs in the aqueous humors: miR-145-5p, miR-302e, miR-1246, and miR-3607-3p; (2) metabolites correlated with miRNAs highly expressed in the culture supernatant of degenerated cells of corneal endothelial cells: miR-23a-3p, miR-24-3p, miR-92b-5p, and miR-184; and (3) metabolites correlated with miRNAs under-expressed in the same degenerated cells: miR-34a-5p, miR-378a-3p, miR-29a-3p, and miR-184 were extracted, and the molecular species shown in FIG. 47 were selected.

1. A diagnosing method for the risk of an ocular disease development or for the disease progression of an end-stage ocular disease by molecular profile analysis of the ocular anterior tissue microenvironment, comprising a step of measuring the concentration level of a metabolite appearing in the collected aqueous humor specimens by mass spectrometry (MS).

2. The diagnosing method according to claim 1, wherein the ocular disease is glaucoma.

3. The diagnosing method according to claim 1, wherein the collected aqueous humor specimens are from a subject in a pre-disease stage with no clinically abnormal signs of ocular disease without identification of the pathological characteristics of the ocular disease.

4. The diagnosing method according to claim 1, wherein the mass spectrometry (MS) is gas chromatography-mass spectrometry (GC-MS) and/or liquid chromatography-mass spectrometry (LC-MS).

5. The diagnosing method according to claim 1, wherein the metabolite is contained in extracellular vesicle (EVs) in the aqueous humors.

6. The diagnosing method according to claim 1, further comprising a step of selecting a metabolite that shows different molecular dynamics from that in tear fluid or blood.

7. The diagnosing method according to claim 2, wherein glaucoma is primary open-angle glaucoma (POAG), normal tension glaucoma (NTG), or pseudoexfoliative glaucoma/pseudoexfoliative syndrome (PEG).

8. The diagnosing method according to claim 2, further comprising a step of discriminating the distinct disease pathogenesis peculiar to each of the three types of glaucoma: primary open-angle glaucoma (POAG), normal tension glaucoma (NTG), and pseudoexfoliative glaucoma/pseudoexfoliative syndrome (PEG).

9. The diagnosing method according to claim 2, wherein the metabolite is selected from those common or not common with the bias observed in subjects with corneal endothelial failures.

10. The diagnosing method according to claim 2, wherein the metabolite is at least one selected from the group consisting of sugar or polyol, a product in the citric acid cycle, acylcarnitine, polyamine, amino acid, and cAMP.

11. The diagnosing method according to claim 10, wherein the sugar or polyol is arabinonic acid, myo-inositol, or fructose; the product in the citric acid cycle is citric acid or isocitric acid; the acylcarnitine is carnitine, isobutyrylcarnitine (C4), or propionylcarnitine; the polyamine is spermidine; and the amino acid is asy-dimethylarginine, quinolinic acid, cysteine, or 3-methylhistidine.

12. The diagnosing method according to claim 10, wherein the sugar or polyol is arabinonic acid or myo-inositol; the acylcarnitine is isobutyrylcarnitine (C4); and the amino acid is cysteine.

13. The diagnosing method according to claim 2, wherein the metabolite is at least one selected from the group consisting of 2-aminoadipic acid, mannose, GSH (glutathione), alanine, spermine, asparagine, choline, glutamine, glutamic acid, pyroglutamic acid, acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formyl kynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.

14. The diagnosing method according to claim 2, wherein the glaucoma is normal tension glaucoma (NTG); and the metabolite is at least one selected from the group consisting 2-aminoadipic acid or mannose, or arabinonic acid, myo-inositol, cAMP, fructose, asy-dimethylarginine, citric acid, quinolinic acid, cysteine, spermidine, carnitine, isobutyrylcarnitine (C4), 3-methylhistidine, propionylcarnitine, and isocitric acid.

15. The diagnosing method according to claim 2, wherein the glaucoma is pseudoexfoliative glaucoma/pseudoexfoliative syndrome (PEG); and the metabolite is selected from the group consisting acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formylkynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.

16. The diagnosing method according to claim 1, wherein the diagnosis is to discriminate among the occurrence of retinal ganglion cell degeneration or the preceding susceptibility of retinal ganglion cells to degeneration stress or cell death, or the vulnerability of retinal ganglion cells.

17. The diagnosing method according to claim 1, comprising a step of referring to the result of comprehensive proteome analysis in the molecular profiles of proteinaceous molecules contained in extracellular vesicle (EVs) in the aqueous humors.

18. The diagnosing method according to claim 1, comprising a step of referring to findings in proteome analysis and/or gene expression analysis using clinical specimens.

19. The diagnosing method according to claim 1, comprising a step of referring to information pertaining to factors in tear fluid, blood, and/or other body fluids.

20. A diagnosing marker for evaluating the risk of ocular disease development or for diagnosing the disease progression of end-stage ocular diseases, which is used in the diagnosing method according to claim 1.

21. The diagnosing marker according to claim 20, which is at least one selected from the group consisting of sugar or polyol, a product in the citric acid cycle, acylcarnitine, polyamine, amino acid, and cAMP.

22. The diagnosing marker according to claim 21, wherein the sugar or polyol is arabinonic acid, myo-inositol, or fructose; the product in the citric acid cycle is citric acid or isocitric acid; the acylcarnitine is carnitine, isobutyrylcarnitine (C4), or propionylcarnitine; the polyamine is spermidine; and the amino acid is asy-dimethylarginine, quinolinic acid, cysteine, or 3-methylhistidine.

23. The diagnosing marker according to claim 21, wherein the sugar or polyol is arabinonic acid or myo-inositol; the acylcarnitine is isobutyrylcarnitine (C4); and the amino acid is cysteine.

24. The diagnosing marker according to claim 20, which is at least one selected from the group consisting of 2-aminoadipic acid, mannose, arabinonic acid, myo-inositol, cAMP, fructose, asy-dimethylarginine, citric acid, quinolinic acid, cysteine, spermidine, carnitine, isobutyrylcarnitine (C4), 3-methylhistidine, propionylcarnitine, GSH (glutathione), alanine, spermine, asparagine, choline, glutamine, glutamic acid, pyroglutamic acid, acetylcholine, xanthosine, N-acetylarginine, glycine, 3-aminoisobutyric acid, cystine, kynurenine, kynurenic acid, 4-hydroxyproline, pyridoxic acid, isocitric acid, N-acetylglucosamine, GSSG (glutathione disulfide), N′-formylkynurenine, creatinine, N-acetylmethionine, ornithine, citrulline, oleamide, arabitol, adenosylhomocysteine, hippuric acid, trans-urocanic acid, urea, succinic acid, riboflavin, 2-hydroxyglutaric acid, malic acid, hypotaurine, pipecolinic acid, guanidinoacetic acid, acetylcarnosine, 2-oxoglutaric acid, 3-hydroxyisovaleric acid, maltose, uridine, 1,5-anhydro-D-sorbitol, fucose, and 2-aminoethanol.

25. A diagnosing system for the risk of an ocular disease development or for the disease progression of an end-stage ocular disease comprising a means for collecting aqueous humor specimens and a means for analyzing the specimens by mass spectrometry (MS), which is used in the diagnosing method according to claim 1.

26. The diagnosing system according to claim 25, wherein the ocular disease is glaucoma.

27. The diagnosing system according to claim 25, wherein the ocular disease is corneal endothelial failures.
